(19) United States
(12) Patent Application Publication
(10) Pub. No.: US 2023/0301580 A1
(43) Pub. Date: Sep. 28, 2023

(54) EAR-WORN DEVICES WITH OROPHARYNGEAL EVENT DETECTION

(71) Applicant: Starkey Laboratories, Inc., Eden Prairie, MN (US)

(72) Inventors: Jinjun Xiao, Chanhassen, MN (US); Amit Shahar, Hod HaSharon (IL)

(21) Appl. No.: 18/018,433

(22) PCT Filed: Jul. 28, 2021
(86) PCT No.: PCT/US2021/043471

§ 371 Date: Jan. 27, 2023

Related U.S. Application Data
(60) Provisional application No. 63/057,722, filed on Jul. 28, 2020.
(60) Provisional application No. 63/058,936, filed on Jul. 30, 2020.

(51) Int. Cl. A61B 5/00; G16H 15/00; A61B 5/11

(52) U.S. Cl. CPC A61B 5/4205; A61B 5/6817; A61B 5/746; G16H 15/00; A61B 5/4866; A61B 5/11; A61B 2562/0219; A61B 2562/0271

(57) ABSTRACT

Embodiments herein relate to ear-worn devices and related systems and methods that can be used detect oropharyngeal events and related occurrences such as food and drink intake. In an embodiment, an ear-worn device system is included having a first ear-worn device that has a control circuit, a motion sensor, at least one microphone, an electroacoustic transducer, and a power supply circuit. The system can also include a second ear-worn device. The system can be configured to monitor signals from at least one of the motion sensor and the at least one microphone and evaluate the signals to identify oropharyngeal events and/or related occurrences. Other embodiments are also included herein.

This application is being filed as a PCT International Patent application on Jul. 28, 2021, in the name of Starkey Laboratories, Inc., a U.S. national corporation, applicant for the designation of all countries, and Jinjun Xiao, a U.S. Citizen, and Amit Shahar, a U.S. Citizen, inventor(s) for the designation of all countries, and claims priority to U.S. Provisional Patent Application No. 63/057,722, filed Jul. 28, 2020, and U.S. Provisional Patent Application No. 63/058,936, filed Jul. 30, 2020, the contents of which are herein incorporated by reference in their entirety.

### FIELD

Embodiments herein relate to ear-worn devices and related systems and methods that can be used detect oropharyngeal events and related occurrences such as food and drink intake.

### BACKGROUND

Food intake is an important factor when considering the health condition of a subject. As such, many people try to track their food intake carefully so that they can evaluate their eating habits and make corrections where necessary. Similarly, clinicians find significant value in tracking their patients' eating habits and try to help their patients establish health eating habits. Liquid intake is also very important for health. Insufficient liquid intake may lead to dehydration, which in turn can lead to many serious complications. For example, dehydration can lead to heat injury, cerebral edema, seizures, hypovolemic shock, kidney failure, coma and even death.

Significant data regarding the health condition of a patient can also be gathered by tracking events associated with eating and drinking. For example, swallowing is a multiphase process that most health subjects can perform effortlessly. However, various conditions (both acute and chronic) may lead to difficulty swallowing. As such, tracking subjects swallowing events can lead to very useful health data.

### SUMMARY

Embodiments herein relate to ear-worn devices and related systems and methods that can be used detect oropharyngeal events and related occurrences such as food and drink intake. In a first aspect, an ear-worn device system is included having a first ear-worn device. The first ear-worn device can include a control circuit, and a motion sensor, wherein the motion sensor is in electrical communication with the control circuit. The first ear-worn device can include at least one microphone, wherein the at least one microphone is in electrical communication with the control circuit and an electroacoustic transducer, wherein the electroacoustic transducer is in electrical communication with the control circuit. The first ear-worn device can also include a power supply circuit, wherein the power supply circuit is in electrical communication with the control circuit. The system can also include a second ear-worn device, wherein the ear-worn device system is configured to monitor signals from at least one of the motion sensor and the at least one microphone, and evaluate the signals to identify oropharyngeal events.

In a second aspect, in addition to one or more of the preceding or following aspects, or in the alternative to some aspects, the oropharyngeal event is selected from the group consisting of mastication, swallowing, and aspiration.

In a third aspect, in addition to one or more of the preceding or following aspects, or in the alternative to some aspects, the system further can include an external device.

In a fourth aspect, in addition to one or more of the preceding or following aspects, or in the alternative to some aspects, the external device can include a smart phone.

In a fifth aspect, in addition to one or more of the preceding or following aspects, or in the alternative to some aspects, the external device can receive data from at least one of the first ear-worn device and the second ear-worn device and evaluates the data to identify an oropharyngeal event.

In a sixth aspect, in addition to one or more of the preceding or following aspects, or in the alternative to some aspects, identification weighting is dependent on a current time of day.

In a seventh aspect, in addition to one or more of the preceding or following aspects, or in the alternative to some aspects, signal evaluation to identify oropharyngeal events includes evaluating signals from the motion sensor to identify signals between 20 and 100 Hz.

In an eighth aspect, in addition to one or more of the preceding or following aspects, or in the alternative to some aspects, signal evaluation to identify oropharyngeal events includes evaluating signals from the microphone to identify signals between 0 and 1.5 kHz.

In a ninth aspect, in addition to one or more of the preceding or following aspects, or in the alternative to some aspects, signal evaluation to identify oropharyngeal events includes evaluating signals from the microphone of the first ear-worn device and signals from a microphone of the second ear-worn device and selecting those signals emanating spatially from a location that is laterally between the first ear-worn device and the second ear-worn device.

In a tenth aspect, in addition to one or more of the preceding or following aspects, or in the alternative to some aspects, signal evaluation to identify oropharyngeal events includes evaluating signals from the microphone of the first ear-worn device and signals from a microphone of the second ear-worn device and selecting those signals emanating from a spatial location that is laterally between the first ear-worn device and the second ear-worn device and posterior to the lips of the ear-worn device wearer.

In an eleventh aspect, in addition to one or more of the preceding or following aspects, or in the alternative to some aspects, the ear-worn device system is configured to evaluate the signals from the motion sensor to identify when the device wearer sits down.

In a twelfth aspect, in addition to one or more of the preceding or following aspects, or in the alternative to some aspects, the ear-worn device system is configured to evaluate the signals from the motion sensor to identify when the device wearer tips their head backward.

In a thirteenth aspect, in addition to one or more of the preceding or following aspects, or in the alternative to some aspects, signal evaluation to identify oropharyngeal events includes evaluating signals from the motion sensor followed sequentially by evaluating signals from the microphone.

In a fourteenth aspect, in addition to one or more of the preceding or following aspects, or in the alternative to some aspects, signal evaluation to identify oropharyngeal events includes evaluating signals from the motion sensor to identify head or jaw movement followed sequentially by evaluating signals from the microphone to identify swallowing.

In a fifteenth aspect, in addition to one or more of the preceding or following aspects, or in the alternative to some aspects, signal evaluation to identify oropharyngeal events includes evaluating signals from the motion sensor and evaluating signals from the at least one microphone to identify mastication using signals from both sensors.

In a sixteenth aspect, in addition to one or more of the preceding or following aspects, or in the alternative to some aspects, signal evaluation to identify oropharyngeal events includes detecting a meal event based on detecting a cluster of mastication events.

In a seventeenth aspect, in addition to one or more of the preceding or following aspects, or in the alternative to some aspects, the ear-worn device system is configured to identify a skipped meal based on the absence of detecting a meal event and a time window for meals.

In an eighteenth aspect, in addition to one or more of the preceding or following aspects, or in the alternative to some aspects, the ear-worn device system is configured to calculate a meal variability score.

In a nineteenth aspect, in addition to one or more of the preceding or following aspects, or in the alternative to some aspects, signal evaluation to identify oropharyngeal events includes binary mastication detection per second.

In a twentieth aspect, in addition to one or more of the preceding or following aspects, or in the alternative to some aspects, the motion sensor can include an accelerometer.

In a twenty-first aspect, in addition to one or more of the preceding or following aspects, or in the alternative to some aspects, the motion sensor can include a gyroscope.

In a twenty-second aspect, in addition to one or more of the preceding or following aspects, or in the alternative to some aspects, the at least one microphone is configured to be positioned within an ear canal of the ear-worn device wearer.

In a twenty-third aspect, in addition to one or more of the preceding or following aspects, or in the alternative to some aspects, the ear-worn device system is configured to distinguish bruxism from mastication.

In a twenty-fourth aspect, in addition to one or more of the preceding or following aspects, or in the alternative to some aspects, the ear-worn device system is configured to distinguish normal swallowing events from abnormal swallowing events.

In a twenty-fifth aspect, in addition to one or more of the preceding or following aspects, or in the alternative to some aspects, the ear-worn device system is configured to distinguish normal swallowing events from abnormal swallowing events based on a sequence of identified oropharyngeal events.

In a twenty-sixth aspect, in addition to one or more of the preceding or following aspects, or in the alternative to some aspects, the ear-worn device system is configured to distinguish normal swallowing events from abnormal swallowing events based on a timing of identified oropharyngeal events.

In a twenty-seventh aspect, in addition to one or more of the preceding or following aspects, or in the alternative to some aspects, the ear-worn device system is configured to generate an alert if abnormal oropharyngeal events are detected crossing a threshold value.

In a twenty-eighth aspect, in addition to one or more of the preceding or following aspects, or in the alternative to some aspects, the ear-worn device system is configured to generate an alert if abnormal oropharyngeal events are detected at a frequency that is increasing over time.

In a twenty-ninth aspect, in addition to one or more of the preceding or following aspects, or in the alternative to some aspects, the ear-worn device system is configured to generate an alert if mastication is detected after a predetermined time.

In a thirtieth aspect, in addition to one or more of the preceding or following aspects, or in the alternative to some aspects, the system further can include a geolocation sensor.

In a thirty-first aspect, in addition to one or more of the preceding or following aspects, or in the alternative to some aspects, the ear-worn device system is configured to generate a report showing geolocations of eating events.

In a thirty-second aspect, in addition to one or more of the preceding or following aspects, or in the alternative to some aspects, the ear-worn device system is configured to generate a report showing time patterns of eating events.

In a thirty-third aspect, in addition to one or more of the preceding or following aspects, or in the alternative to some aspects, the ear-worn device system is configured to generate a report showing frequency of fluid intake.

In a thirty-fourth aspect, in addition to one or more of the preceding or following aspects, or in the alternative to some aspects, the ear-worn device system is configured to generate an alert if the frequency of fluid intake falls below a fluid intake threshold value. In some embodiments, the alert can take the form of a prompt for the device wearer to drink more fluids or a prompt for a third-party to administer fluids to the device wearer.

In a thirty-fifth aspect, in addition to one or more of the preceding or following aspects, or in the alternative to some aspects, the fluid intake threshold value is a predetermined static value.

In a thirty-sixth aspect, in addition to one or more of the preceding or following aspects, or in the alternative to some aspects, the fluid intake threshold value is a dynamically determined value.

In a thirty-seventh aspect, in addition to one or more of the preceding or following aspects, or in the alternative to some aspects, the at least one microphone can include a front microphone, and a rear microphone.

In a thirty-eighth aspect, in addition to one or more of the preceding or following aspects, or in the alternative to some aspects, the first ear-worn device is configured to detect food types.

In a thirty-ninth aspect, in addition to one or more of the preceding or following aspects, or in the alternative to some aspects, the ear-worn device system is configured to estimate food intake quantities.

In a fortieth aspect, in addition to one or more of the preceding or following aspects, or in the alternative to some aspects, the ear-worn device system is configured to estimate calorie intake.

In a forty-first aspect, in addition to one or more of the preceding or following aspects, or in the alternative to some aspects, the first ear-worn device can include a temperature sensor.

In a forty-second aspect, in addition to one or more of the preceding or following aspects, or in the alternative to some aspects, the at least one microphone can include an intracanal microphone.

In a forty-third aspect, in addition to one or more of the preceding or following aspects, or in the alternative to some aspects, the at least one microphone can include a pair of intracanal microphones.

In a forty-fourth aspect, an ear-worn device is included having a first ear-worn device, the first ear-worn device can include a control circuit, a motion sensor, wherein the motion sensor is in electrical communication with the control circuit, at least one microphone, wherein the at least one microphone is in electrical communication with the control circuit, an electroacoustic transducer, wherein the electroacoustic transducer is in electrical communication with the control circuit, and a power supply circuit, wherein the power supply circuit is in electrical communication with the control circuit, wherein the ear-worn device is configured to monitor signals from at least one of the motion sensor and the at least one microphone, and evaluate the signals to identify oropharyngeal events.

In a forty-fifth aspect, in addition to one or more of the preceding or following aspects, or in the alternative to some aspects, the oropharyngeal event is selected from the group consisting of mastication, swallowing, and aspiration.

In a forty-sixth aspect, in addition to one or more of the preceding or following aspects, or in the alternative to some aspects, signal evaluation to identify oropharyngeal events includes evaluating signals from the motion sensor to identify signals between 20 and 100 Hz.

In a forty-seventh aspect, in addition to one or more of the preceding or following aspects, or in the alternative to some aspects, signal evaluation to identify oropharyngeal events includes evaluating signals from the microphone to identify signals between 0 and 1.5 kHz.

In a forty-eighth aspect, in addition to one or more of the preceding or following aspects, or in the alternative to some aspects, the ear-worn device is configured to evaluate the signals from the motion sensor to identify when the device wearer sits down.

In a forty-ninth aspect, in addition to one or more of the preceding or following aspects, or in the alternative to some aspects, the ear-worn device is configured to evaluate the signals from the motion sensor to identify when the device wearer tips their head backward.

In a fiftieth aspect, in addition to one or more of the preceding or following aspects, or in the alternative to some aspects, signal evaluation to identify oropharyngeal events includes evaluating signals from the motion sensor followed sequentially by evaluating signals from the microphone.

In a fifty-first aspect, in addition to one or more of the preceding or following aspects, or in the alternative to some aspects, signal evaluation to identify oropharyngeal events includes evaluating signals from the motion sensor to identify head or jaw movement followed sequentially by evaluating signals from the microphone to identify swallowing.

In a fifty-second aspect, in addition to one or more of the preceding or following aspects, or in the alternative to some aspects, signal evaluation to identify oropharyngeal events includes evaluating signals from the motion sensor and evaluating signals from the at least one microphone to identify mastication using signals from both sensors.

In a fifty-third aspect, in addition to one or more of the preceding or following aspects, or in the alternative to some aspects, signal evaluation to identify oropharyngeal events includes detecting a meal event based on detecting a cluster of mastication events.

In a fifty-fourth aspect, in addition to one or more of the preceding or following aspects, or in the alternative to some aspects, signal evaluation to identify oropharyngeal events includes binary mastication detection per second.

In a fifty-fifth aspect, in addition to one or more of the preceding or following aspects, or in the alternative to some aspects, the motion sensor can include an accelerometer.

In a fifty-sixth aspect, in addition to one or more of the preceding or following aspects, or in the alternative to some aspects, the motion sensor can include a gyroscope.

In a fifty-seventh aspect, in addition to one or more of the preceding or following aspects, or in the alternative to some aspects, at least one microphone is configured to be positioned within an ear canal of the ear-worn device wearer.

In a fifty-eighth aspect, in addition to one or more of the preceding or following aspects, or in the alternative to some aspects, the ear-worn device is configured to distinguish bruxism from mastication.

In a fifty-ninth aspect, in addition to one or more of the preceding or following aspects, or in the alternative to some aspects, the ear-worn device is configured to distinguish normal swallowing events from abnormal swallowing events.

In a sixtieth aspect, in addition to one or more of the preceding or following aspects, or in the alternative to some aspects, the ear-worn device is configured to distinguish normal swallowing events from abnormal swallowing events based on a sequence of identified oropharyngeal events.

In a sixty-first aspect, in addition to one or more of the preceding or following aspects, or in the alternative to some aspects, the ear-worn device is configured to distinguish normal swallowing events from abnormal swallowing events based on a timing of identified oropharyngeal events.

In a sixty-second aspect, in addition to one or more of the preceding or following aspects, or in the alternative to some aspects, the ear-worn device is configured to generate an alert if abnormal oropharyngeal events are detected crossing a threshold value.

In a sixty-third aspect, in addition to one or more of the preceding or following aspects, or in the alternative to some aspects, the ear-worn device is configured to generate an alert if abnormal oropharyngeal events are detected at a frequency that is increasing over time.

In a sixty-fourth aspect, in addition to one or more of the preceding or following aspects, or in the alternative to some aspects, the device further can include a geolocation sensor.

In a sixty-fifth aspect, in addition to one or more of the preceding or following aspects, or in the alternative to some aspects, the ear-worn device is configured to generate a report showing geolocations of eating events.

In a sixty-sixth aspect, in addition to one or more of the preceding or following aspects, or in the alternative to some aspects, the ear-worn device is configured to generate a report showing time patterns of eating events.

In a sixty-seventh aspect, in addition to one or more of the preceding or following aspects, or in the alternative to some aspects, the ear-worn device is configured to generate a report showing frequency of fluid intake.

In a sixty-eighth aspect, in addition to one or more of the preceding or following aspects, or in the alternative to some aspects, the ear-worn device is configured to generate an alert if the frequency of fluid intake falls below a fluid intake threshold value.

In a sixty-ninth aspect, in addition to one or more of the preceding or following aspects, or in the alternative to some aspects, the fluid intake threshold value is a predetermined static value.

In a seventieth aspect, in addition to one or more of the preceding or following aspects, or in the alternative to some aspects, the fluid intake threshold value is a dynamically determined value.

In a seventy-first aspect, in addition to one or more of the preceding or following aspects, or in the alternative to some aspects, the ear-worn device is configured to generate an alert if mastication is detected after a predetermined time.

In a seventy-second aspect, in addition to one or more of the preceding or following aspects, or in the alternative to some aspects, the at least one microphone can include a front microphone, and a rear microphone.

In a seventy-third aspect, an ear-worn device system is included having a first ear-worn device, the first ear-worn device can include a control circuit, a motion sensor, wherein the motion sensor is in electrical communication with the control circuit, at least one microphone, wherein the at least one microphone is in electrical communication with the control circuit, an electroacoustic transducer, wherein the electroacoustic transducer is in electrical communication with the control circuit, and a power supply circuit, wherein the power supply circuit is in electrical communication with the control circuit, wherein the ear-worn device system is configured to monitor signals from at least one of the motion sensor and the at least one microphone, and transfer data representing the signals to an external device for identification of oropharyngeal events.

In a seventy-fourth aspect, in addition to one or more of the preceding or following aspects, or in the alternative to some aspects, the oropharyngeal event is selected from the group consisting of mastication, swallowing, and aspiration.

In a seventy-fifth aspect, in addition to one or more of the preceding or following aspects, or in the alternative to some aspects, signal evaluation to identify oropharyngeal events includes evaluating signals from the motion sensor to identify signals between 20 and 100 Hz.

In a seventy-sixth aspect, in addition to one or more of the preceding or following aspects, or in the alternative to some aspects, signal evaluation to identify oropharyngeal events includes evaluating signals from the microphone to identify signals between 0 and 1.5 kHz.

In a seventy-seventh aspect, in addition to one or more of the preceding or following aspects, or in the alternative to some aspects, the ear-worn device system is configured to evaluate the signals from the motion sensor to identify when the device wearer sits down.

In a seventy-eighth aspect, in addition to one or more of the preceding or following aspects, or in the alternative to some aspects, the ear-worn device system is configured to evaluate the signals from the motion sensor to identify when the device wearer tips their head backward.

In a seventy-ninth aspect, in addition to one or more of the preceding or following aspects, or in the alternative to some aspects, signal evaluation to identify oropharyngeal events includes evaluating signals from the motion sensor followed sequentially by evaluating signals from the microphone.

In an eightieth aspect, in addition to one or more of the preceding or following aspects, or in the alternative to some aspects, signal evaluation to identify oropharyngeal events includes evaluating signals from the motion sensor to identify head or jaw movement followed sequentially by evaluating signals from the microphone to identify swallowing.

In a eighty-first aspect, in addition to one or more of the preceding or following aspects, or in the alternative to some aspects, signal evaluation to identify oropharyngeal events includes evaluating signals from the motion sensor and evaluating signals from the at least one microphone to identify mastication using signals from both sensors.

In a eighty-second aspect, in addition to one or more of the preceding or following aspects, or in the alternative to some aspects, signal evaluation to identify oropharyngeal events includes detecting a meal event based on detecting a cluster of mastication events.

In a eighty-third aspect, in addition to one or more of the preceding or following aspects, or in the alternative to some aspects, signal evaluation to identify oropharyngeal events includes binary mastication detection per second.

In a eighty-fourth aspect, in addition to one or more of the preceding or following aspects, or in the alternative to some aspects, the motion sensor can include an accelerometer.

In a eighty-fifth aspect, in addition to one or more of the preceding or following aspects, or in the alternative to some aspects, the motion sensor can include a gyroscope.

In a eighty-sixth aspect, in addition to one or more of the preceding or following aspects, or in the alternative to some aspects, the at least one microphone is configured to be positioned within an ear canal of the ear-worn device wearer.

In a eighty-seventh aspect, in addition to one or more of the preceding or following aspects, or in the alternative to some aspects, the ear-worn device system is configured to distinguish bruxism from mastication.

In a eighty-eighth aspect, in addition to one or more of the preceding or following aspects, or in the alternative to some aspects, the ear-worn device system is configured to distinguish normal swallowing events from abnormal swallowing events.

In a eighty-ninth aspect, in addition to one or more of the preceding or following aspects, or in the alternative to some aspects, the ear-worn device system is configured to distinguish normal swallowing events from abnormal swallowing events based on a sequence of identified oropharyngeal events.

In a ninetieth aspect, in addition to one or more of the preceding or following aspects, or in the alternative to some aspects, the ear-worn device system is configured to distinguish normal swallowing events from abnormal swallowing events based on a timing of identified oropharyngeal events.

In a ninety-first aspect, in addition to one or more of the preceding or following aspects, or in the alternative to some aspects, the ear-worn device system is configured to generate an alert if abnormal oropharyngeal events are detected crossing a threshold value.

In a ninety-second aspect, in addition to one or more of the preceding or following aspects, or in the alternative to some aspects, the ear-worn device system is configured to generate an alert if abnormal oropharyngeal events are detected at a frequency that is increasing over time.

In a ninety-third aspect, in addition to one or more of the preceding or following aspects, or in the alternative to some aspects, the ear-worn device system is configured to generate an alert if mastication is detected after a predetermined time.

In a ninety-fourth aspect, in addition to one or more of the preceding or following aspects, or in the alternative to some aspects, the device or system can include a geolocation sensor.

In a ninety-fifth aspect, in addition to one or more of the preceding or following aspects, or in the alternative to some aspects, the ear-worn device system is configured to generate a report showing geolocations of eating events.

In a ninety-sixth aspect, in addition to one or more of the preceding or following aspects, or in the alternative to some aspects, the ear-worn device system is configured to generate a report showing time patterns of eating events.

In a ninety-seventh aspect, in addition to one or more of the preceding or following aspects, or in the alternative to some aspects, the ear-worn device system is configured to generate a report showing frequency of fluid intake.

In a ninety-eighth aspect, in addition to one or more of the preceding or following aspects, or in the alternative to some aspects, the ear-worn device system is configured to generate an alert if the frequency of fluid intake falls below a fluid intake threshold value.

In a ninety-ninth aspect, in addition to one or more of the preceding or following aspects, or in the alternative to some aspects, the fluid intake threshold value is a predetermined static value.

In a one hundredth aspect, in addition to one or more of the preceding or following aspects, or in the alternative to some aspects, the fluid intake threshold value is a dynamically determined value.

In a one hundred and first aspect, in addition to one or more of the preceding or following aspects, or in the alternative to some aspects, the external device can include a smart phone.

In a one hundred and second aspect, in addition to one or more of the preceding or following aspects, or in the alternative to some aspects, the at least one microphone can include: a front microphone, and a rear microphone.

In a one hundred and third aspect, a method of detecting oropharyngeal events with an ear-worn device is included. The method can include monitoring signals from at least one of a motion sensor associated with the ear-worn device and a microphone associated with the ear-worn device, and evaluating the signals to identify an oropharyngeal event.

In a one hundred and fourth aspect, in addition to one or more of the preceding or following aspects, or in the alternative to some aspects, evaluating the signals to identify an oropharyngeal event further includes evaluating signals of both the motion sensor and the microphone.

In a one hundred and fifth aspect, in addition to one or more of the preceding or following aspects, or in the alternative to some aspects, the method further can include processing the signals.

In a one hundred and sixth aspect, in addition to one or more of the preceding or following aspects, or in the alternative to some aspects, processing the signals further includes filtering out motion sensor signals below 20 Hz.

In a one hundred and seventh aspect, in addition to one or more of the preceding or following aspects, or in the alternative to some aspects, processing the signals further includes correlating signals from at least two microphones to extract spatially defined signals.

In a one hundred and eighth aspect, in addition to one or more of the preceding or following aspects, or in the alternative to some aspects, the spatially defined signals include those with a determined point of origin within the ear-worn device wearer.

In a one hundred and ninth aspect, in addition to one or more of the preceding or following aspects, or in the alternative to some aspects, processing the signals further includes exacting at least one spectral feature of the microphone signal.

In a one hundred and tenth aspect, in addition to one or more of the preceding or following aspects, or in the alternative to some aspects, processing the signals further includes exacting at least one temporal feature of the microphone signal.

In a one hundred and eleventh aspect, in addition to one or more of the preceding or following aspects, or in the alternative to some aspects, processing the signals further includes filtering out signals corresponding to the voice of the ear-worn device wearer and the voices of third parties.

In a one hundred and twelfth aspect, in addition to one or more of the preceding or following aspects, or in the alternative to some aspects, the method further can include calculating a trend based on identified oropharyngeal events.

In a one hundred and thirteenth aspect, in addition to one or more of the preceding or following aspects, or in the alternative to some aspects, the method further can include issuing an alert if an abnormal oropharyngeal event is identified.

In a one hundred and fourteenth aspect, in addition to one or more of the preceding or following aspects, or in the alternative to some aspects, the oropharyngeal event includes mastication.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following FIGURES (FIGS.), in which:

FIG. 1 is a diagram illustrating some aspects of human anatomy relevant to oropharyngeal events that can be detected and/or tracked with devices and system herein.

FIG. 2 is a schematic view of an ear-worn device shown in accordance with various embodiments herein.

FIG. 3 is a partial cross-sectional view of ear anatomy shown in accordance with various embodiments herein.

FIG. 4 is a schematic view of an ear-worn device disposed within the ear of a ear-worn device wearer in accordance with various embodiments herein.

FIG. 5 is a schematic frontal view of a subject wearing ear-worn devices in accordance with various embodiments herein.

FIG. 6 is a schematic side view of a subject wearing an ear-worn device in accordance with various embodiments herein.

FIG. 7 is a schematic view of data and/or signal flow as part of a system in accordance with various embodiments herein.

FIG. 8 is a diagram of an exemplary process for detecting oropharyngeal events such as chewing using data from a microphone shown in accordance with various embodiments herein.

FIG. 9 is a diagram of an exemplary process for detecting oropharyngeal events such as chewing using data from an IMU is shown in accordance with various embodiments herein.

FIG. 10 is a diagram illustrating oropharyngeal events, and specifically mastication associated with eating across the days of a week in accordance with various embodiments herein.

FIG. 11 is a diagram illustrating various parameters that may be tracked over time in order to determine one or more trends regarding an ear-worn device wearer's behavior/status in accordance with various embodiments herein.

FIG. 12 is a schematic block diagram of various components of an ear-worn device in accordance with various embodiments.

FIG. 13 is a graph showing separation of sound signals in accordance with various embodiments herein.

FIG. 14 is a graph showing separation of sound signals in accordance with various embodiments herein.

FIG. 15 is a graph showing motion sensor signals and the identification of oropharyngeal events therein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

As referenced above, food intake and liquid intake are important factors to track in order to monitor a subject's health condition. In addition, there is significant value in tracking events associated with eating and drinking, such as swallowing.

However, tracking such things is difficult for many people, particularly where they must track things manually. In addition, even for people who track such information, it is difficult to keep information that is complete enough to provide care providers and clinicians with a complete picture of the subject's eating and drinking habits.

In addition, people may not be fully cognizant of their swallowing events and how they might have changed over time. Even if they are aware, they may have trouble describing things in sufficient detail so as to be actionable by a care provider or clinician.

The normal adult swallowing process includes four phases. The first phase is the oral preparatory phase. The second phase is the oral transit phase. The third phase is the pharyngeal phase. The fourth phase is the esophageal phase.

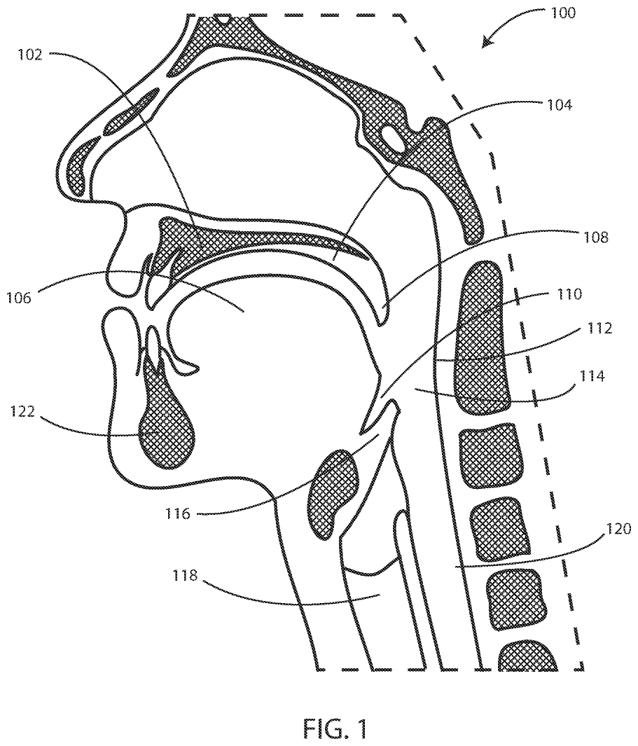

Referring now to FIG. 1, some aspects of human anatomy relevant to oropharyngeal events are illustrated in cross-section and described with respect to the role them play during the four phases of a normal swallowing process. During the first phase (oral preparatory phase) food is manipulated through chewing (mastication) into a cohesive unit referred to as a bolus. In specific, food is chewed and mixed with saliva to form the bolus and then the bolus is positioned on the tongue 106 for transport. It has been found herein that movement of the jaw 122 during this phase associated with mastication creates movement and sounds that can be detected using sensors of embodiments herein, including, but not limited to, motion sensors and microphones.

During the second phase (oral transit phase) the bolus is moved back through the mouth with a front-to-back squeezing action that is performed primarily by the tongue 106, which moves upward and forward with the tip of the tongue contacting the hard palate 102. The tongue-palate contact area expands in a backward direction causing the bolus to be pushed into the oral pharynx 114 and the valleculae 110 (space between the epiglottis 116 and the back of the tongue). The jaw 122 then assumes a downward position and the tongue drops away from the palate. In a normal scenario, this phase takes approximately one second to perform. Movements and sound characteristic of this phase can be detected using sensors of embodiments herein, including, but not limited to, motion sensors and microphones.

During the third phase (pharyngeal phase) the food enters the upper throat area, the soft palate 104 and uvula 108 elevates, and the epiglottis 116 closes off the trachea 118 as the tongue 106 moves backward and the pharyngeal wall 112 moves forward. The bolus is forced downward to the esophagus 120 and then breathing is reinitiated. In normal scenarios, this phase can also take approximately one second to perform. Movements and sound characteristic of this phase can be detected using sensors of embodiments herein, including, but not limited to, motion sensors and microphones.

During the fourth phase (esophageal phase) the food bolus enters the esophagus 120 and then is moved through the esophagus 120 and to the stomach by a squeezing action of the throat muscles.

Aspiration is the occurrence of inhalation of liquids, food materials, stomach contents, or secretions into the lungs. Specifically, aspiration occurs when such materials enter the trachea instead of entering the esophagus. Normally, very small quantities of materials are aspirated and mechanisms such as coughing and lung cilia can effectively remove the materials. However, problems with the nervous system, musculoskeletal system, or respiratory system may result in quantities of materials being aspirated that can lead to serious health problems. Symptoms of aspiration can include choking, coughing, wheezing, throat clearing, unexplained low-grade fever, wet/gurgling voice, respiratory changes, low oxygen saturation, fatigue, and the like. Aspiration can also lead to aspiration pneumonia which is a very serious condition. Aspiration pneumonia can quickly get worse if not properly diagnosed and treated. Movements and sound characteristic of aspiration and various symptoms related thereto can be detected using sensors of embodiments herein, including, but not limited to, motion sensors and microphones.

Ear-worn devices (or “ear-wearable” devices) herein and related systems can be used to detect oropharyngeal events (both normal and abnormal) including, but not limited to, mastication, swallowing, drinking, aspiration, and the like. As such, embodiments herein include ear-worn devices and related systems that can be used to track aspects such as eating, drinking, swallowing, and other oropharyngeal events. In some embodiments, an exemplary a first ear-worn device can include a control circuit, a motion sensor, one or more microphones, an electroacoustic transducer, and a power supply circuit. The ear-worn device system can be configured to monitor signals from at least one of the motion sensor and the microphone and evaluate the signals to identify oropharyngeal events.

The term “ear-worn device” as used herein shall refer to devices that can aid a person with impaired hearing. The term “ear-worn device” shall also refer to devices that can produce optimized or processed sound for persons with normal hearing. Ear-worn devices herein can include hearing assistance devices. Ear-worn devices herein can include, but are not limited to, behind-the-ear (BTE), in-the ear (ITE), in-the-canal (ITC), invisible-in-canal (IIC), receiver-in-canal (RIC), receiver in-the-ear (RITE) and completely-in-the-canal (CIC) type hearing assistance devices. In some embodiments, the ear-worn device can be a hearing aid falling under 21 C.F.R. § 801.420. In another example, the ear-worn device can include one or more Personal Sound Amplification Products (PSAPs). In another example, the ear-worn device can include one or more cochlear implants, cochlear implant magnets, cochlear implant transducers, and cochlear implant processors. In another example, the ear-worn device can include one or more “hearable” devices that provide various types of functionality. In other examples, ear-worn devices can include other types of devices that are wearable in, on, or in the vicinity of the user's ears. In other examples, ear-worn devices can include other types of devices that are implanted or otherwise osseointegrated with the user's skull, wherein the device is able to facilitate stimulation of the wearer's ears via the bone conduction pathway.

Ear-worn devices herein, including hearing aids and hearables (e.g., wearable earphones), can include an enclosure, such as a housing, shell or other structure, within which internal components are disposed. Many different components can be included. In some embodiments, components of an ear-worn device herein can include at least one of a control circuit, digital signal processor (DSP), memory (such as non-volatile memory), power management circuitry, a data communications bus, one or more communication devices (e.g., a radio, a near-field magnetic induction device), one or more antennas, one or more microphones, a receiver/speaker, and various sensors as described in greater detail below. More advanced ear-worn devices can incorporate a long-range communication device, such as a BLUETOOTH® transceiver or other type of radio frequency (RF) transceiver.

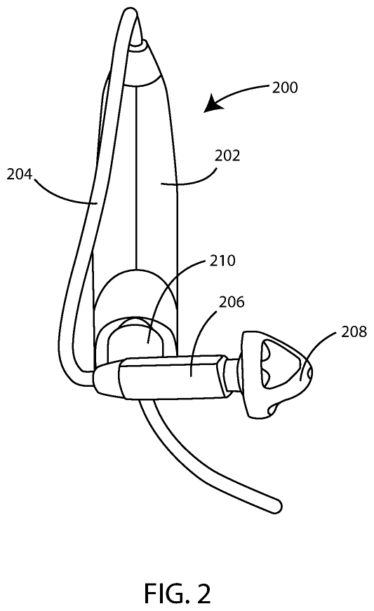

Referring now to FIG. 2, a schematic view of an ear-worn device 200 is shown in accordance with various embodiments herein. The ear-worn device 200 can include a hearing device housing 202. The hearing device housing 202 can define a battery compartment 210 into which a battery can be disposed to provide power to the device. The ear-worn device 200 can also include a receiver 206 adjacent to an earbud 208. The receiver 206 an include a component that converts electrical impulses into sound, such as an electroacoustic transducer, speaker, or loud speaker. Such components can be used to generate an audible stimulus in various embodiments herein. A cable 204 or connecting wire can include one or more electrical conductors and provide electrical communication between components inside of the hearing device housing 202 and components inside of the receiver 206.

In some embodiments, sensors as described herein, such as motion sensors, microphones, and the like can be disposed on or within hearing device housing 202, which could be on the ear. In some embodiments, sensors as described herein, such as motion sensors, microphones, and the like can be disposed on or within hearing receiver 206, which could be within the ear canal. In some embodiments, sensors can be in both places and/or other locations.

The ear-worn device 200 shown in FIG. 2 is a receiver-in-canal type device and thus the receiver is designed to be placed within the ear canal. However, it will be appreciated that many different form factors for ear-worn devices are contemplated herein. As such, ear-worn devices herein can include, but are not limited to, behind-the-ear (BTE), in-the ear (ITE), in-the-canal (ITC), invisible-in-canal (IIC), receiver-in-canal (RIC), receiver in-the-ear (RITE) and completely-in-the-canal (CIC) type hearing assistance devices.

Ear-worn devices of the present disclosure can incorporate an antenna arrangement coupled to a high-frequency radio, such as a 2.4 GHz radio. The radio can conform to an IEEE 802.11 (e.g., WIFI®) or BLUETOOTH® (e.g., BLE, BLUETOOTH® 4.2 or 5.0) specification, for example. It is understood that ear-worn devices of the present disclosure can employ other radios, such as a 900 MHz radio. Ear-worn devices of the present disclosure can be configured to receive streaming audio (e.g., digital audio data or files) from an electronic or digital source. Representative electronic/digital sources (also referred to herein as accessory devices) include an assistive listening system, a TV streamer, a radio, a smartphone, a cell phone/entertainment device (CPED) or other electronic device that serves as a source of digital audio data or files.

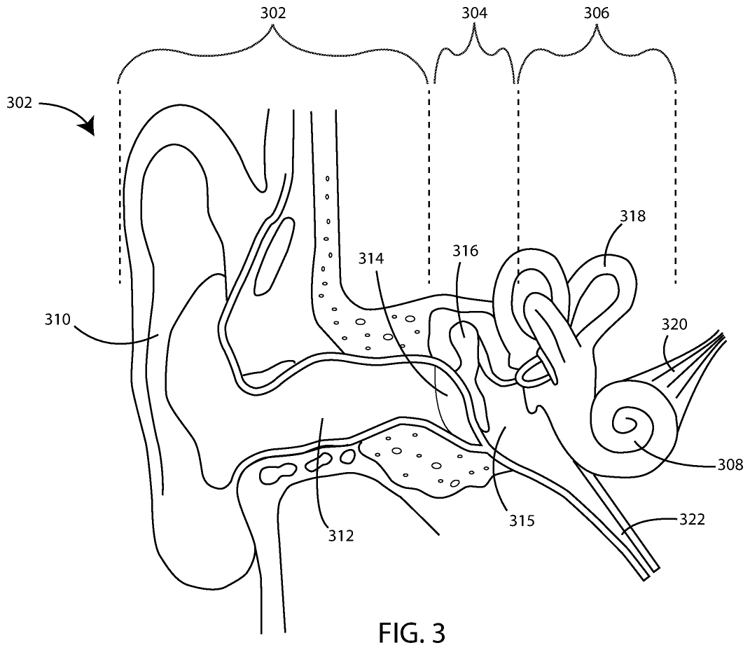

Referring now to FIG. 3, a partial cross-sectional view of ear anatomy is shown. The three parts of the ear anatomy are the outer ear 302, the middle ear 304 and the inner ear 306. The inner ear 306 includes the cochlea 308. (‘Cochlea’ means ‘snail’ in Latin; the cochlea gets its name from its distinctive coiled up shape.) The outer ear 302 includes the pinna 310, ear canal 312, and the tympanic membrane 314 (or eardrum). The middle ear 304 includes the tympanic cavity 315, auditory bones 316 (malleus, incus, stapes), and facial nerve. The inner ear 306 includes the cochlea 308, and the semicircular canals 318, and the auditory nerve 320. The pharyngotympanic tube 322 is in fluid communication with the eustachian tube and helps to control pressure within the middle ear generally making it equal with ambient air pressure.

Sound waves enter the ear canal 312 and make the tympanic membrane 314 vibrate. This action moves the tiny chain of auditory bones 316 (ossicles—malleus, incus, stapes) in the middle ear 304. The last bone in this chain contacts the membrane window of the cochlea 308 and makes the fluid in the cochlea 308 move. The fluid movement then triggers a response in the auditory nerve 320.

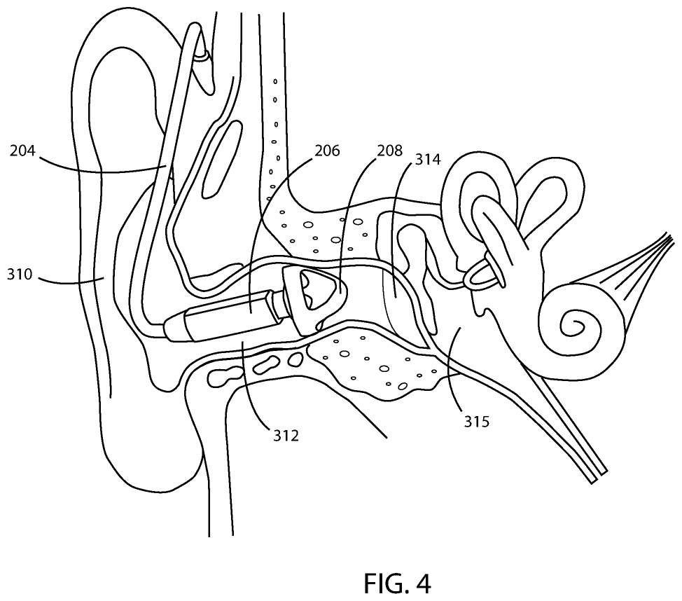

As mentioned above, the ear-worn device 200 shown in FIG. 2 can be a receiver-in-canal type device and thus the receiver is designed to be placed within the ear canal. Referring now to FIG. 4, a schematic view is shown of an ear-worn device disposed within the ear of a subject in accordance with various embodiments herein. In this view, the receiver 206 and the earbud 208 are both within the ear canal 312, but do not directly contact the tympanic membrane 314. The hearing device housing is mostly obscured in this view behind the pinna 310, but it can be seen that the cable 204 passes over the top of the pinna 310 and down to the entrance to the ear canal 312.

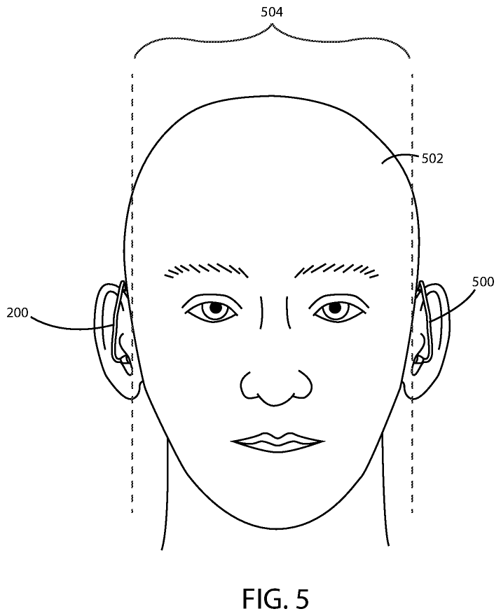

Referring now to FIG. 5, a schematic frontal view is shown of a subject 502 wearing ear-worn devices 200, 500 in accordance with various embodiments herein. FIG. 5 also illustrates a middle zone 504 that is between the first ear-worn device 200 and the second ear-worn device 500. The middle zone 504 is the area in which sound and motion relevant for oropharyngeal events originates. In various embodiments herein, devices and systems can be configured for sensitivity to sound and/or motion originating in the middle zone 504 between the first ear-worn device 200 and the second ear-worn device 500. In various embodiments herein, devices and systems can be configured to distinguish between sounds originating in the middle zone 504 and sounds originating outside of the middle zone 504.

In various embodiments, signal evaluation or processing to identify oropharyngeal events can include evaluating signals from the microphone of the first ear-worn device and signals from a microphone of the second ear-worn device and selecting those signals emanating spatially from a location that is laterally between the first ear-worn device and the second ear-worn device.

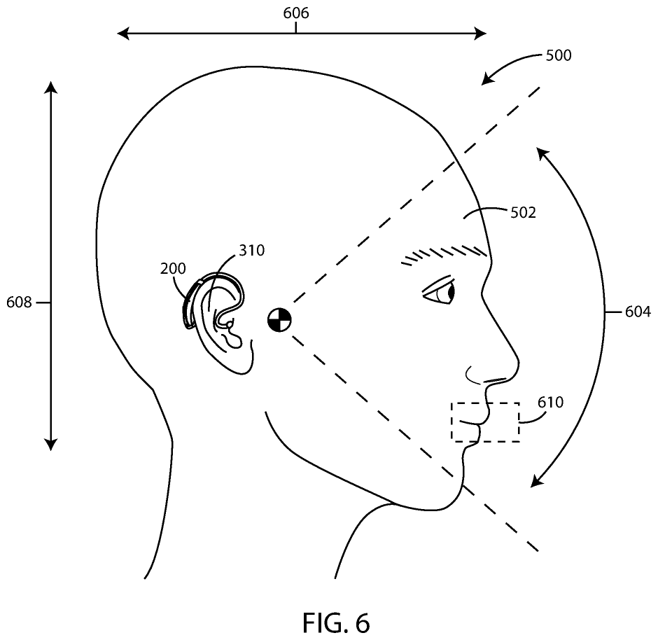

Ear-worn devices herein can include sensors (such as part of a sensor package) to detect movements of the subject wearing the ear-worn device. Referring now to FIG. 6, a schematic side view is shown of a subject 502 wearing an ear-worn device 200 in accordance with various embodiments herein. For example, movements detected can include forward/back movements 606, up/down movements 608, and rotational movements 604 in the vertical plane, as well as in the horizontal plane, amongst others.

Certain oropharyngeal events such as drinking are frequently accompanied by a characteristic head movement immediately prior to the event. For example, an individual commonly tips their head backward before beginning to drink from a glass. In some embodiments, ear-worn device systems herein are configured to evaluate the signals from a motion sensor to identify when the device wearer tips their head backward. In some embodiments, signal evaluation to identify oropharyngeal events includes evaluating signals from the motion sensor followed sequentially by evaluating signals from the microphone. In some embodiments, signal evaluation to identify oropharyngeal events includes evaluating signals from the motion sensor to identify head or jaw movement followed sequentially by evaluating signals from the microphone to identify eating, drinking, or swallowing.

In some embodiments, weighting factors for identification of oropharyngeal events can vary depending on whether another event is detected. For example, weighting factors can be changed such that signals from one or more microphones, motion sensors, or other sensors occurring immediately after head or jaw movement characteristic of the device wearer putting food in their mouth or bringing a drink to their lips are more likely to be deemed an oropharyngeal event than are signals from the sensors in the absence of such head or jaw movements.

In various embodiments, devices and systems herein can be configured to distinguish between sounds originating at or near a sound origin 610 associated with speech versus sounds originating at other points within the body of the subject 502. In an embodiment, signal evaluation or processing to identify oropharyngeal events can include evaluating signals from the microphone of the first ear-worn device and signals from a microphone of the second ear-worn device and selecting those signals emanating from a spatial location that is laterally between the first ear-worn device and the second ear-worn device and posterior to the lips of the ear-worn device wearer.

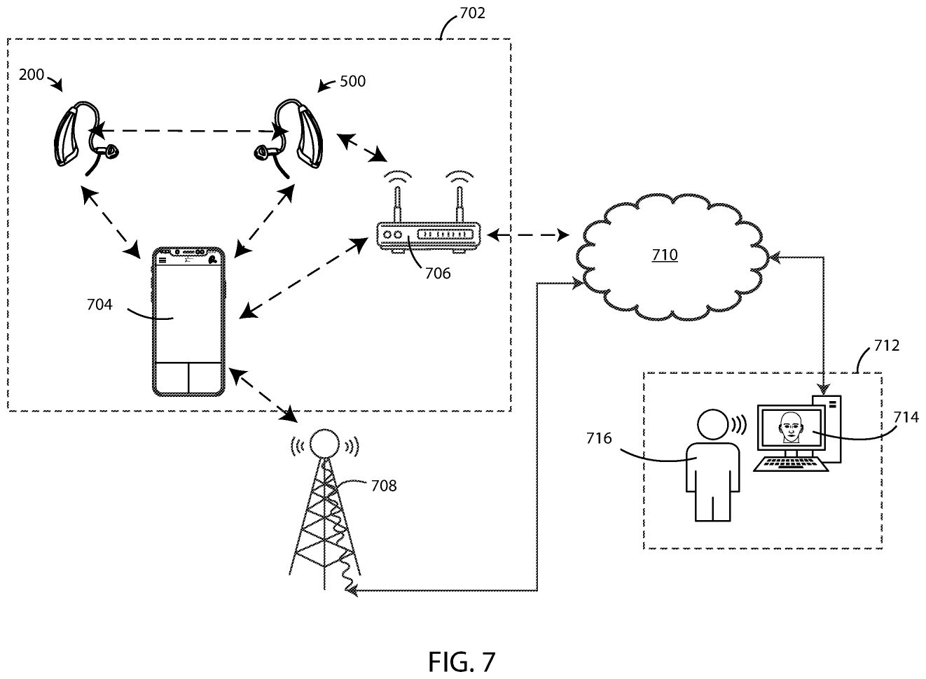

It will be appreciated that data and/or signals can be exchanged between many different components in accordance with embodiments herein. Referring now to FIG. 7, a schematic view is shown of data and/or signal flow as part of a system in accordance with various embodiments herein. In a first location 702, a device wearer (not shown) can have a first ear-worn device 200 and a second ear-worn device 500. Each of the ear-worn devices 200, 500 can include sensor packages as described herein including, for example, an IMU and one or more microphones. The ear-worn devices 200, 500 and sensors therein can be disposed on opposing lateral sides of the subject's head. In some embodiments, the ear-worn devices 200, 500 and sensors therein can be disposed in a fixed position relative to the subject's head. The ear-worn devices 200, 500 and sensors therein can be disposed, at least partially, within opposing ear canals of the subject. The ear-worn devices 200, 500 and sensors therein can be disposed on or in opposing ears of the subject. The ear-worn devices 200, 500 and sensors therein can be spaced apart from one another by a distance of at least 3, 4, 5, 6, 8, 10, 12, 14, or 16 centimeters and less than 40, 30, 28, 26, 24, 22, 20 or 18 centimeters, or by a distance falling within a range between any of the foregoing.

In various embodiments, data and/or signals can be exchanged directly between the first ear-worn device 200 and the second ear-worn device 500. An external visual display device 704 (or external device) with a video display screen, such as a smart phone, can also be disposed within the first location 702. The external visual display device 704 can exchange data and/or signals with one or both of the first ear-worn device 200 and the second ear-worn device 500 and/or with an accessory to the ear-worn devices (e.g., a remote microphone, a remote control, a phone streamer, etc.). The external visual display device 704 can also exchange data across a data network to the cloud 710, such as through a wireless signal connecting with a local gateway device, such as a network router 706, mesh network, or through a wireless signal connecting with a cell tower 708 or similar communications tower. In some embodiments, the external visual display device can also connect to a data network to provide communication to the cloud 710 through a direct wired connection.

In some embodiments, a care provider 716 (such as an audiologist, physical therapist, a physician or a different type of clinician, specialist, or care provider, or physical trainer) can receive information remotely at a second location 712 from device(s) at the first location 702 through a data communication network such as that represented by the cloud 710. The care provider 716 can use a computing device 714 to see and interact with the information received. The received information can include, but is not limited to, information regarding oropharyngeal events of the subject such as eating events, drinking events, swallowing, and the like. In some embodiments, received information can be provided to the care provider 716 in real time. In some embodiments, received information can be stored and provided to the care provider 716 at a time point after response times are measured.

In some embodiments, the care provider 716 (such as an audiologist, physical therapist, a physician or a different type of clinician, specialist, or care provider, or physical trainer) can send information remotely from the second location 712 through a data communication network such as that represented by the cloud 710 to devices at the first location 702. For example, the care provider 716 can enter information into the computing device 714, can use a camera connected to the computing device 714 and/or can speak into the external computing device. The sent information can include, but is not limited to, feedback information, guidance information, and the like. In some embodiments, feedback information from the care provider 716 can be provided to the subject in real time.

As such, embodiments herein can include operations of sending data to a remote system user at a remote site, receiving feedback from the remote system user, and presenting the feedback to the subject. In some cases, the feedback can be auditory. The operation of presenting the auditory feedback to the subject can be performed with the ear-worn device(s). In various embodiments, the operation of presenting the auditory feedback to the subject can be performed with an ear-worn device(s).

Ear-worn devices of the present disclosure can incorporate an antenna arrangement coupled to a high-frequency radio, such as a 2.4 GHz radio. The radio can conform to an IEEE 802.11 (e.g., WIFI®) or BLUETOOTH® (e.g., BLE, BLUETOOTH® 4.2 or 5.0) specification, for example. It is understood that ear-worn devices of the present disclosure can employ other radios, such as a 900 MHz radio or radios operating at other frequencies or frequency bands. Ear-worn devices of the present disclosure can be configured to receive streaming audio (e.g., digital audio data or files) from an electronic or digital source. Representative electronic/digital sources (also referred to herein as accessory devices) include an assistive listening system, a TV streamer, a radio, a smartphone, a cell phone/entertainment device (CPED) or other electronic device that serves as a source of digital audio data or files. Systems herein can also include these types of accessory devices as well as other types of devices.

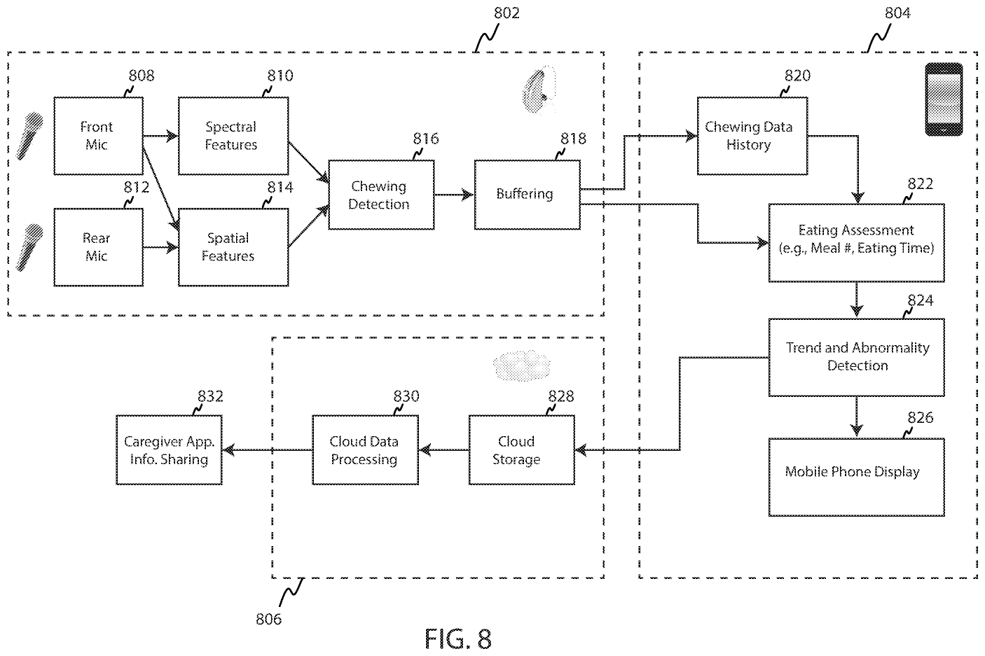

Referring now to FIG. 8, a diagram of an exemplary process for detecting oropharyngeal events such as chewing using data from a microphone is shown in accordance with various embodiments herein. In this example, various operations are performed at the level of an ear-worn device 802, an external device 804, and the cloud 806.

A front microphone 808 is used to generate signals representative of sound along with a rear microphone 812. In this example, the signals from the front microphone 808 are then processed in order to evaluate/extract spectral and/or temporal features 810 therefrom. Many different spectral and/or temporal features can be evaluated/extracted including, but not limited to, those shown in the following table.

TABLE 1
Feature Name

Zero-Crossing Rate
Periodicity Strength
Short Time Energy
Spectral Centroid
Spectral Centroid Mean
Spectral Bandwidth
Spectral Roll-off
Spectral Flux
High-/Low-Frequency Energy Ratio
High-Frequency Slope
Low-Frequency Slope
Absolute Magnitude Difference Function
Spectral Flux at High Frequency
Spectral Flux at Low Frequency
Periodicity Strength
Low Frequency Envelope Peakiness
Onset Rate

Spectral and/or temporal features that can be utilized from signals of a single-mic can include, but are not limited to, HLF (the relative power in the high-frequency portion of the spectrum relative to the low-frequency portion), SC (spectral centroid), LS (the slope of the power spectrum below the Spectral Centroid), PS (periodic strength), and Envelope Peakiness (a measure of signal envelope modulation).

In embodiments with at least two microphones, one or more of the following signal features can be used to detect mastication (chewing) using the spatial information between two microphones.
MSC: Magnitude Squared Coherence.
ILD: level difference
IPD: phase difference

The MSC feature can be used to determine whether a source is a point source or distributed. The ILD and IPD features can be used to determine the direction of arrival of the sound. Chewing sounds are located at a particular location relative to the microphones on the device. Also chewing sounds are distributed and caused by chewing activities in the whole mouth (in contrast, for example, speech is mostly emitted from the lips.)

It will be appreciated that when at least two microphones are used that have some physical separation from one another that the signals can then be processed to derive/extract/utilize spatial information 814. For example, signals from the front microphone 808 and the rear microphone 812 can be correlated in order to extract those signals representing sound with a point of origin falling in an area associated with the inside of the device wearer. As such, this operation can be used to separate signals associated with external noise and external speech from signals associated with oropharyngeal sounds of the device wearer.

Using data associated with the sensor signals directly, spectral features of the sensor signals, and/or data associated with spatial features, an operation can be executed in order to detect 816 an oropharyngeal event such as mastication (chewing), swallowing, aspiration, and the like.

In some embodiments, various machine learning techniques can be applied in order to take the signals from sensors as described herein and determine whether or not a oropharyngeal event has occurred. In specific, machine learning techniques related to classification can be applied in order to determine whether or not oropharyngeal events have occurred. Machine learning techniques that can be applied can include supervised learning, unsupervised learning, and reinforcement learning. In some embodiments, techniques applied can include one or more of artificial neural networks, convolutional neural networks, K nearest neighbor techniques, decision trees, support vector machines, or the like. In some embodiments herein, a multi-node decision tree can be used to reach a binary result (e.g. binary classification) on whether the individual is chewing or not.

In some embodiments, signals or other data derived therefrom can be divided up into discrete time units (such as periods of milliseconds, seconds, minutes, or longer) and the system can perform binary classification (e.g., “eating” or “not eating”) regarding whether the individual was eating during that discrete time unit. As an example, in some embodiments, signal processing or evaluation operations herein to identify oropharyngeal events can include binary classification for mastication detection on a per second basis.

In some cases, other types of data can also be evaluated when identifying an oropharyngeal event. For example, in some cases the ear-worn device ear-worn device system can be configured to evaluate the signals from a motion sensor or other sensor to identify when the device wearer sits down. The process of sitting down includes a characteristic pattern that can be identified from evaluation of a motion sensor signal. Weighting factors for identification of an oropharyngeal event can be adjusted if the system detects that the individual has sat down, since most meals are consumed while individuals are seated.

In some embodiments, weighting factors for identification of oropharyngeal events can vary depending on whether the device wearer is detected to have assumed a seated position. For example, weighting factors can be changed such that signals from one or more microphones, motion sensors, or other sensors occurring while the device wearer is sitting down are more likely to be deemed an oropharyngeal event than are signals from the sensors while the device wearer is standing, walking, or lying down.

In some embodiments, the system can be configured to distinguish bruxism (teeth grinding) from mastication (chewing). This can be accomplished in various ways. Because teeth grinding is more likely to occur while sleeping, in one approach, aspects of timing and the posture of the ear-worn device wearer can be taken into account by the system. For example, using the motion sensor and/or components thereof such as an accelerometer or a gyroscope it can be determined whether or not the device wearer is lying down. Since eating while lying down is very uncommon, weight factors can be adjusted so as to result in the system determining that an oropharyngeal event such as mastication has not taken place.

Abnormal oropharyngeal events can be detected by the system in various ways. For example, in some embodiments, repetitive swallowing can be used to detect abnormal oropharyngeal events. For example, if an individual attempts to swallow something but is not successful, they may (through voluntary or involuntary means) attempt to swallow again rapidly and this can be identified because there may not be sufficient time between swallowing attempts for it to represent a normal sequence of swallowing as outlined above. Thus, rapid repeated swallowing can be used by the system in order to identify an abnormal oropharyngeal event such as unsuccessful and/or incomplete swallowing. In some embodiments, the ear-worn device system can be configured to distinguish normal swallowing events from abnormal swallowing events based on a timing of identified oropharyngeal events.

In some embodiments, detecting of coughing, gagging, aspiration or breathing cessation immediately after detection of mastication and swallowing can be used to identify an abnormal oropharyngeal event. Coughing, gagging, aspiration, and similar events can be detected using signals from sensors such as microphones and motion sensors using techniques similar to those used to detect oropharyngeal events as described elsewhere herein. Thus, in some embodiments the ear-worn device system can be configured to distinguish normal swallowing events from abnormal swallowing events based on a sequence of identified oropharyngeal events.

In some embodiments, data regarding oropharyngeal events can be buffered 818 at the level of the ear-worn device 802 before being passed on to an external device 804.

While FIG. 8 illustrates detection of an oropharyngeal event at the level of the ear-worn device 802, it will be appreciated that in some embodiments signal data can be passed from the ear-worn device 802 onto the external device 804 and detection can occur there.

At the level of the external device 804, oropharyngeal event data history can be stored 820. In addition, an eating assessment/evaluation can be performed 822, which can include calculation of various aspects such as the number of meals per unit time (such as per day, week, or month), eating times, average eating duration, eating locations, numbers of skipped meals, and the like.

In various embodiments, an operation of trend and abnormality calculation 824 can be performed. Trends can include, but are not limited to, eating time trend, eating duration trend, eating location trend, eating day of the week trend, and the like. Abnormalities can include, but are not limited to, abnormal eating times, abnormal eating durations, abnormal eating locations, abnormal oropharyngeal events detected (repetitive swallowing, incomplete swallowing, aspiration, coughing, etc.) and the like. In various embodiments, information is then displayed 826 for the subject to see the determined information.

Then, in some embodiments, at least some data can be passed to the cloud 806 and, specifically, be subject to storage 828 and/or further processing operations 830.

In some embodiments, data can then be based to an application for a caregiver 832 in order to share information with them that may be relevant for the care they provide to the subject. In some cases, the data provided to the caregiver can include any of the trend or abnormality data referred to above. In some cases, the data provided to the caregiver can include any data recorded regarding any oropharyngeal event.

While FIG. 8 exemplifies a scenario with an ear-worn device having two microphones (a front microphone and a rear microphone), it will be appreciated that similar techniques can be applied to ear-worn devices having different numbers of microphones. In addition, in some embodiments, similar techniques can be applied to a system including two ear-worn devices (e.g., a right and a left device) with the correlation (spatial isolation) occurring between signals of each ear-worn device (versus between signals of microphones of the same ear-worn device).

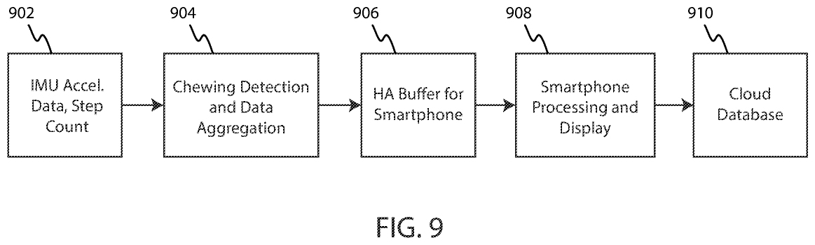

Referring now to FIG. 9, a diagram of an exemplary process for detecting oropharyngeal events such as chewing using data from an IMU is shown in accordance with various embodiments herein.

In a first operation 902, IMU data (such as accelerometer data) is gathered. In some embodiments, step count data is also gathered. Step count data can be derived from accelerometer data and/or can be received from a different component or device that is either part of the ear-worn device system or part of a different system.

In a second operation 904, the IMU data is processed in order to detect oropharyngeal events, such as mastication (chewing). In the second operation 904, data regarding or related to oropharyngeal events can also be aggregated.

In a third operation 906, data can be transferred or buffered and transferred to an external device, such as a smartphone. In a fourth operation 908, the external device can be used in order to process and/or display data regarding oropharyngeal events. In a fifth operation 910, data regarding oropharyngeal events can be passed on to a cloud database for storage.

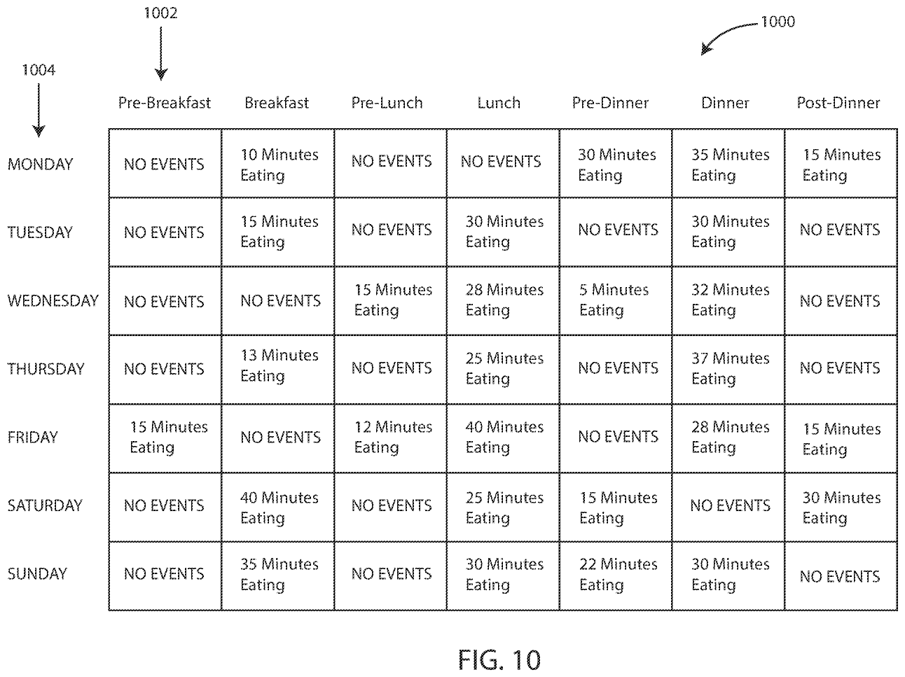

Referring now to FIG. 10, a diagram 1000 is shown illustrating oropharyngeal events, and specifically mastication associated with eating across the days of a week. In this example, a plurality of time windows 1002 are shown having relevance toward food intake. Specifically, in this example, hours of a day are broken up into time windows of pre-breakfast, breakfast, pre-lunch, lunch, pre-dinner, dinner, and post-dinner. In some embodiments, these time windows 1002 can correspond to specific hours of the day according to default rules (e.g., the breakfast is from 7 AM to 9 AM, lunch is from 11:00 to 1:00, and dinner is from 5:00 to 7:30 PM). However, in some embodiments, these time windows 1002 can be set according to input from the ear-worn device wearer and/or a clinician, care provider, or other third party. In some embodiments, these time windows can be dynamically set by the system itself in accordance with past observations of when the ear-worn device wearer typically eats. For example, data from a given time period (for example, an initial four weeks of time) can be processed according to machine learning techniques in order to derive time windows 1002 that match the exhibited behavior of the ear-worn device wearer.

In some embodiments, the total amount of time spent eating within each time window can be tracked and/or displayed. In some embodiments, an amount of time spent eating that crosses a threshold value can be counted as a meal. In some embodiments, detecting a meal event is based on detecting a cluster of mastication events. In some embodiments, detecting a meal event is based on detecting a threshold number of mastication events within a fixed time period, such as within 1, 2, 3, 4, 5, 7, 10, or 15 minutes, or an amount of time falling within a range between any of the foregoing.

In some embodiments, the system can be configured to identify a skipped meal based on the absence of detecting a meal event during a time window for meals. For example, if no meal is detected during the breakfast time window, then that can be counted as an occurrence of a skipped meal. In some embodiments, the system can be configured to identify a skipped meal based on the total number of meals detected during a given day. For example, if only two meals are detected on a given day, then that can be counted as an occurrence of a skipped meal.

Based on detected timing of meals, the detected length of meals, the detected locations of meals, the detected food type(s) of meals, and/or the detected calories within meals the ear-worn device system can be configured to calculate a meal variability score.

In some embodiments, the system can be configured to detect food types. For example, the system can be configured to distinguish crunchier foods such as vegetables and fruits from starch-heavy foods such as pasta and grains based on signals from the motion sensor, microphone or other sensors reflecting the crunchiness of the food being eaten. In some embodiments, the system is configured to estimate food intake quantities. Such food intake estimates can be performed in various ways. As one example, the duration of food intake time can be used to estimate the quantity of food intake. In some embodiments, the system can be configured to estimate calorie intake. In some embodiments, calorie intake can be estimated using detected food types and estimated food intake quantities.

In the example of FIG. 10, the time periods 1004 for display are days of the week. However, it will be appreciated that the time periods 1004 could be any period of time such as days, weeks, months, years, or the like. In some embodiments, the total amount of time eating and/or the total number of meals can be tracked across the days of the week.

While not shown in this view, data regarding the physical location at which mastication/eating/meals can be tracked. For example, on a given day dinner may be eaten at a restaurant while other dinners of the week may be eaten at home. The locations of the restaurant and the home location can be stored for purposes of tracking eating locations. In some embodiments, a report showing eating locations can be generated by the system.

In some embodiments, weighting factors for identification of oropharyngeal events can vary depending on the time of the day. For example, weighting factors can be changed such that signals from one or more microphones, motion sensors, or other sensors occurring during a normal time period for meals (such as during a breakfast, lunch or dinner time window) are more likely to be deemed an oropharyngeal event than are signals from the sensors during an abnormal time period for meals (such as in the middle of the night, pre-breakfast, etc.).

Information can be tracked over time and trends can be calculated along with deviations from trends, such as deviations that may represent a change in behavior (good or bad) or a health concern. Various reports and/or alerts can be generated based on the information shown with respect to FIG. 10 and/or other information determined by the system herein. For example, the ear-worn device system is configured to generate an alert if mastication is detected after a predetermined time of the day. As another example, an alert can be generated if a skipped meal is detected. In some embodiments, alerts herein can be an auditory and/or visual alert to the ear-worn device wearer. In some embodiments, the alert can be an electronic communication that is sent from an ear-worn device or other device that is part of the ear-worn system directly to or through a communication network to a clinician, care provider, or other third party. The system can also generate various reports for display to the ear-worn device wearer or to a clinician, care provider, or other third party. In some embodiments, reports or alerts herein and/or the information therefrom can be delivered to a smartphone (or other mobile computing device) companion application. An exemplary companion application is the THRIVE® companion application available from Starkey Hearing Technologies, Eden Prairie, MN. For example, the ear-worn device system can be configured to generate a report showing geolocations of eating events. As another example, the ear-worn device system can be configured to generate a report showing time patterns of eating events.

While FIG. 10 illustrates mastication and meal occurrences, it will be appreciated that similar principles can also be applied to fluid intake events and monitoring of hydration. In some embodiments, the ear-worn device system can be configured to generate a report showing frequency of fluid intake. In some embodiments, the ear-worn device system can be configured to generate an alert if the frequency of fluid intake falls below a fluid intake threshold value. In some embodiments, the ear-worn device system can be configured to generate an alert if an estimated state of hydration of the device wearer balls below a threshold value. In some embodiments, the alert can specifically take the form of a prompt for the device wearer to consume fluids. In some embodiments, the alert can take the form of a prompt for a third party to administer fluids to the device wearer. In some embodiments, the alert can take the form of a prompt for the device wearer to consume a specific amount of fluids. The prompts can be provided in various ways. In some embodiments, a prompt can be delivered through the ear-worn device itself, such as an audio prompt. In some embodiments, the prompt can be delivered through an accessory device or another device, such as in the form of an audio, visual, and/or tactile prompt.

In some scenarios, the fluid intake (or hydration) threshold value can be a predetermined static value. In other scenarios, the fluid intake threshold value is a dynamically determined value taking into account one or more of: the age of the device wearer, the weight of the device wearer, the activity level of the device wearer, ambient temperatures and humidity, and the like.

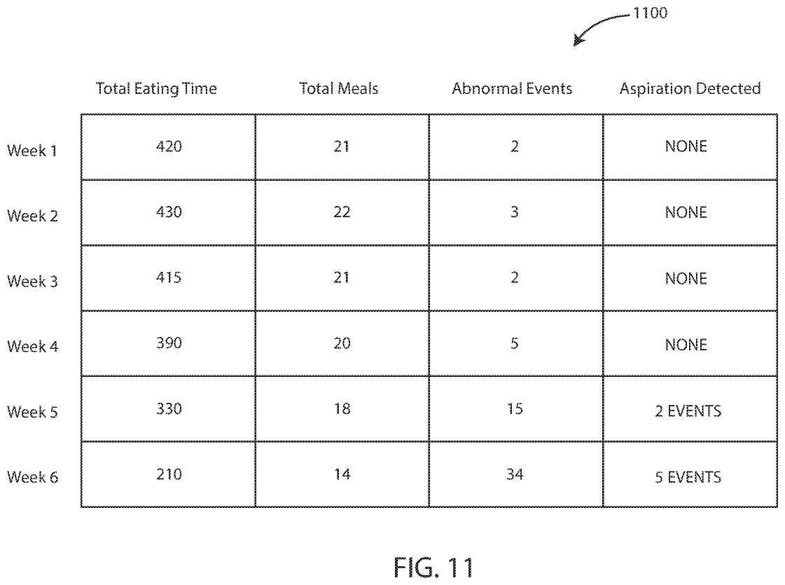

Referring now to FIG. 11, a diagram 1100 is shown illustrating various parameters that may be tracked over time in order to determine one or more trends regarding the ear-worn device wearer's behavior/status. In this example, specific parameters for purposes of evaluating/displaying trend data include total eating time (such as in minutes) per week, total meals per week, total number of abnormal events per week, as well as the number of aspiration events detected per week. It will be appreciated that these parameters are simply provided by way of illustration and that the actual parameters used for evaluating/displaying trend data can include any of the aspects referenced herein as well as other oropharyngeal related events.

In FIG. 11, a trend of decreasing eating time and decreasing number of meals is illustrated that occurs simultaneously with a trend of increasing abnormal events and an increasing trend of aspiration. Any of these trends (as well as many others) may be of sufficient concern in order for the system to generate and/or send an alert. In some embodiments, the alert may be an auditory and/or visual alert to the ear-worn device wearer. In some embodiments, the alert may be an electronic communication that is sent from an ear-worn device or other device that is part of the ear-worn system directly to or through a communication network to a clinician, care provider, or other third party.

Beyond alerts based on trends, in some embodiments, the system can be configured to generate and/or send alerts if any parameters or oropharyngeal event measures cross a threshold value and/or represent a departure from a recent trend. In some embodiments, threshold values can be predetermined. In some embodiments, threshold values can be default values that are preprogrammed into the ear-worn device. In some embodiments, threshold values can be set according to input from the ear-worn device wearer and/or a clinician, care provider, or other third party. In some embodiments, these threshold values can be dynamically set by the system itself in accordance with past observations of the ear-worn device wearer. For example, data from a given time period (for example, an initial four weeks of time) can be processed according to machine learning techniques and/or statistical techniques in order to derive thresholds that are significant for an individual ear-worn device wearer.

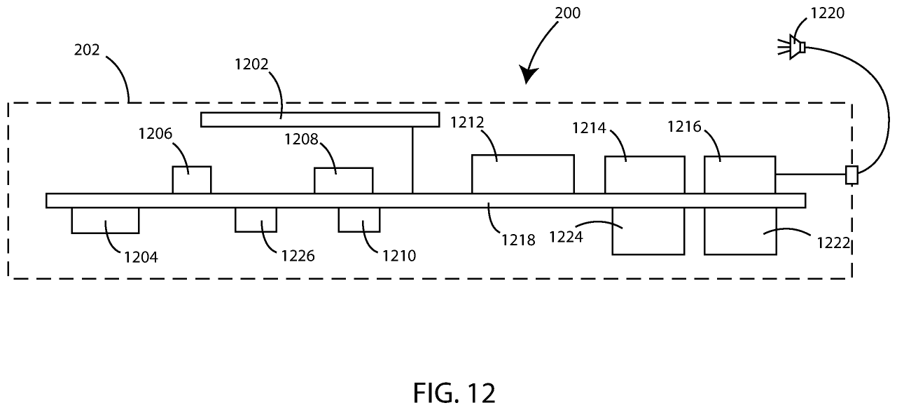

In will be appreciated that ear-worn devices herein can include various components. Referring now to FIG. 12, a schematic block diagram is shown with various components of an ear-worn device in accordance with various embodiments. The block diagram of FIG. 12 represents a generic ear-worn device for purposes of illustration. The ear-worn device 200 shown in FIG. 12 includes several components electrically connected to a flexible mother circuit 1218 (e.g., flexible mother board) which is disposed within housing 202. A power supply circuit 1204 can include a battery and can be electrically connected to the flexible mother circuit 1218 and provides power to the various components of the ear-worn device 200. One or more microphones 1206 are electrically connected to the flexible mother circuit 1218, which provides electrical communication between the microphones 1206 and a digital signal processor (DSP) 1212. Among other components, the DSP 1212 incorporates or is coupled to audio signal processing circuitry configured to implement various functions described herein. A sensor package 1214 can be coupled to the DSP 1212 via the flexible mother circuit 1218. The sensor package 1214 can include one or more different specific types of sensors such as those described in greater detail below. One or more user switches 1210 (e.g., on/off, volume, mic directional settings) are electrically coupled to the DSP 1212 via the flexible mother circuit 1218.

An audio output device 1216 is electrically connected to the DSP 1212 via the flexible mother circuit 1218. In some embodiments, the audio output device 1216 comprises a speaker (coupled to an amplifier). In other embodiments, the audio output device 1216 comprises an amplifier coupled to an external receiver 1220 adapted for positioning within an ear of a wearer. The external receiver 1220 can include an electroacoustic transducer, speaker, or loud speaker. The ear-worn device 200 may incorporate a communication device 1208 coupled to the flexible mother circuit 1218 and to an antenna 1202 directly or indirectly via the flexible mother circuit 1218. The communication device 1208 can be a BLUETOOTH® transceiver, such as a BLE (BLUETOOTH® low energy) transceiver or other transceiver(s) (e.g., an IEEE 802 502.11 compliant device). The communication device 1208 can be configured to communicate with one or more external devices, such as those discussed previously, in accordance with various embodiments. In various embodiments, the communication device 1208 can be configured to communicate with an external visual display device such as a smart phone, a video display screen, a tablet, a computer, or the like.

In various embodiments, the ear-worn device 200 can also include a control circuit 1222 and a memory storage device 1224. The control circuit 1222 can be in electrical communication with other components of the device. In some embodiments, a clock circuit 1226 can be in electrical communication with the control circuit. The control circuit 1222 can execute various operations, such as those described herein. The control circuit 1222 can include various components including, but not limited to, a microprocessor, a microcontroller, an FPGA (field-programmable gate array) processing device, an ASIC (application specific integrated circuit), or the like. The memory storage device 1224 can include both volatile and non-volatile memory. The memory storage device 1224 can include ROM, RAM, flash memory, EEPROM, SSD devices, NAND chips, and the like. The memory storage device 1224 can be used to store data from sensors as described herein and/or processed data generated using data from sensors as described herein.

It will be appreciated that various of the components described in FIG. 12 can be associated with separate devices and/or accessory devices to the ear-worn device. By way of example, microphones can be associated with separate devices and/or accessory devices. Similarly, audio output devices can be associated with separate devices and/or accessory devices to the ear-worn device.

### Sensors

Ear-worn devices as well as medical devices herein can include one or more sensor packages (including one or more discrete or integrated sensors) to provide data. The sensor package can comprise one or a multiplicity of sensors. In some embodiments, the sensor packages can include one or more motion sensors amongst other types of sensors. Motion sensors herein can include inertial measurement units (IMU), accelerometers, gyroscopes, barometers, altimeters, and the like. The IMU can be of a type disclosed in commonly owned U.S. patent application Ser. No. 15/331,230, filed Oct. 21, 2016, which is incorporated herein by reference. In some embodiments, electromagnetic communication radios or electromagnetic field sensors (e.g., telecoil, NFMI, TMR, GME, etc.) sensors may be used to detect motion or changes in position. In some embodiments, biometric sensors may be used to detect body motions or physical activity. Motions sensors can be used to track movement of a patient in accordance with various embodiments herein.

In some embodiments, the motion sensors can be disposed in a fixed position with respect to the head of a patient, such as worn on or near the head or ears. In some embodiments, the operatively connected motion sensors can be worn on or near another part of the body such as on a wrist, arm, or leg of the patient.

According to various embodiments, the sensor package can include one or more of an IMU, and accelerometer (3, 6, or 9 axis), a gyroscope, a barometer, an altimeter, a magnetometer, a magnetic sensor, an eye movement sensor, a pressure sensor, an acoustic sensor, a telecoil, a heart rate sensor, a global positioning system (GPS) or other geolocation circuit, a temperature sensor, a blood pressure sensor, an oxygen saturation sensor, an optical sensor, a blood glucose sensor (optical or otherwise), a galvanic skin response sensor, a cortisol level sensor (optical or otherwise), a microphone, acoustic sensor, an electrocardiogram (ECG) sensor, electroencephalography (EEG) sensor which can be a neurological sensor, eye movement sensor (e.g., electrooculogram (EOG) sensor), myographic potential electrode sensor (EMG), a heart rate monitor, a pulse oximeter, a wireless radio antenna, blood perfusion sensor, hydrometer, sweat sensor, cerumen sensor, air quality sensor, pupillometry sensor, cortisol level sensor, hematocrit sensor, light sensor, image sensor, and the like.

The ear-worn device can include any number of microphones as part of its sensor package. In some embodiments, the ear-worn device can include 1, 2, 3, 4, 5, 6, or more microphones or a number of microphones falling within a range between any of the foregoing. In some embodiments, the ear-worn device can specifically include a front microphone and a rear microphone (with reference to the anterior-posterior axis of the ear-worn device wearer). In some embodiments, microphones herein may be associated with (e.g., disposed on or in) portions of the ear-worn device that are external to the ear canal. In some embodiments, microphones herein can be associated with (e.g., disposed on or in) portions of the ear-worn device that are internal to the ear canal (intracanal microphones). In some embodiments, the set of microphones that are part of an ear-worn device can include those that are external to the ear canal as well as those that are internal to the ear canal.

In some embodiments, the sensor package can be part of an ear-worn device. However, in some embodiments, the sensor packages can include one or more additional sensors that are external to an ear-worn device. For example, various of the sensors described above can be part of a wrist-worn or ankle-worn sensor package, or a sensor package supported by a chest strap.

Data produced by the sensor(s) of the sensor package can be operated on by a processor of the device or system.

As used herein the term “inertial measurement unit” or “IMU” shall refer to an electronic device that can generate signals related to a body's specific force and/or angular rate. IMUs herein can include one or more accelerometers (3, 6, or 9 axis) to detect linear acceleration and a gyroscope to detect rotational rate. In some embodiments, an IMU can also include a magnetometer to detect a magnetic field.

Sensors herein can be of various types. By way of example, a pressure sensor can be, for example, a MEMS-based pressure sensor, a piezo-resistive pressure sensor, a flexion sensor, a strain sensor, a diaphragm-type sensor and the like. A temperature sensor can be, for example, a thermistor (thermally sensitive resistor), a resistance temperature detector, a thermocouple, a semiconductor-based sensor, an infrared sensor, or the like. A blood pressure sensor can be, for example, a pressure sensor. The heart rate sensor can be, for example, an electrical signal sensor, an acoustic sensor, a pressure sensor, an infrared sensor, an optical sensor, or the like. An oxygen saturation sensor (such as a blood oximetry sensor) can be, for example, an optical sensor, an infrared sensor, or the like. An electrical signal sensor can include two or more electrodes and can include circuitry to sense and record electrical signals including sensed electrical potentials and the magnitude thereof (according to Ohm's law where V=IR) as well as measure impedance from an applied electrical potential.

It will be appreciated that the sensor package can include one or more sensors that are external to the ear-worn device. In addition to the external sensors discussed hereinabove, the sensor package can comprise a network of body sensors (such as those listed above) that sense movement of a multiplicity of body parts (e.g., arms, legs, torso). In some embodiments, the ear-worn device can be in electronic communication with the sensors or processor of another medical device, e.g., an insulin pump device, a heart pacemaker device, a wearable device, or the like.

### Methods

Many different methods are contemplated herein, including, but not limited to, methods of making ear worn devices, methods of using ear worn devices to detect oropharyngeal events, and the like. Aspects of system/device operation described elsewhere herein can be performed as operations of one or more methods in accordance with various embodiments herein.

In an embodiment, a method of detecting oropharyngeal events with an ear-worn device is included, the method monitoring signals from at least one of a motion sensor associated with the ear-worn device and a microphone associated with the ear-worn device, and evaluating the signals to identify an oropharyngeal event.

In various embodiments herein, evaluating the signals to identify an oropharyngeal event further comprises evaluating signals of both the motion sensor and the microphone.

In various embodiments herein, one or more operations of processing the signals is performed. It will be appreciated that signal processing can include various operations including, but not limited to, filtering, amplification, feature calculation and/or extraction, and the like. In various embodiments, processing the signals further comprises filtering out motion sensor signals below a threshold of 30, 25, 20, 15, or 10 Hz, or a threshold value falling within a range between any of the foregoing. In various embodiments, processing the signals further comprises filtering out motion sensor signals above a threshold of 70, 80, 90, 100, 110, or 120 Hz, or a threshold value falling within a range between any of the foregoing. In some embodiments, signal evaluation to identify oropharyngeal events includes evaluating signals from the motion sensor to identify signals between 20 and 100 Hz.

In various embodiments herein, processing the signals further comprises filtering out microphone signals corresponding to the voice of the ear-worn device wearer and the voices of third parties.

In some embodiments, signal processing includes filtering out signals from the microphone above a threshold value of 1 kHz, 1.25 kHz, 1.5 kHz, 1.75 kHz, or 2 kHz, or a threshold value falling within a range between any of the foregoing. In some embodiments, signal processing or evaluation to identify oropharyngeal events includes evaluating signals from the microphone to identify signals between 0 and 1.5 kHz.

In various embodiments, processing the signals further comprises extracting at least one spectral feature of the microphone signal. In various embodiments, processing the signals further comprises extracting at least one temporal feature of the microphone signal.

In various embodiments, processing the signals further comprises correlating signals from at least two microphones to extract spatially defined signals. In various embodiments, the spatially defined signals comprise those with a determined point of origin within the ear-worn device wearer.

In various embodiment herein, an operation of calculating a trend based on identified oropharyngeal events can be performed, such as described elsewhere herein with respect to trends.

In various embodiments herein, operations performed can further include issuing an alert if an abnormal oropharyngeal event is identified as described elsewhere herein with respect to alerts.

In various embodiments, an oropharyngeal event detected herein can include at least one of mastication, swallowing, eating, drinking, aspiration, and the like. In some embodiments, the oropharyngeal event specifically includes mastication.

Aspects may be better understood with reference to the following examples. These examples are intended to be representative of specific embodiments, but are not intended as limiting the overall scope of embodiments herein.

### EXAMPLES

### Example 1: Spectral Feature-Based Separation of Sound Components

An individual was fitted with an ear-worn device (MC hearing aid) including a microphone. Sounds were recorded using a single microphone of the individual eating an apple, the individual talking, and another person talking approximately 1 meter away from the microphone. These sounds were then digitally mixed to form a test sound signal.

The test signal was then processed to extract various features thereof. In particular, the test signal was processed to evaluate the following features: various features including the Low Frequency Spectral Peakiness.

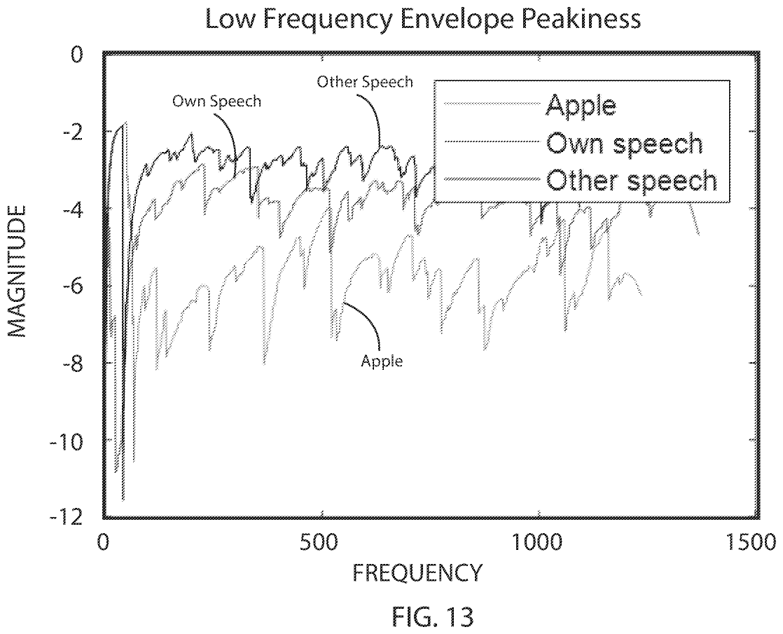

The results for a single feature are shown in FIG. 13. Specifically, FIG. 13 shows separation of sound signals representing eating an apple (“Apple), versus an ear-worn device wearer's own voice (“Own Speech”), versus speech of others (“Other Speech”), using a particular spectral feature (“Low Frequency Spectral Peakiness”). FIG. 13 shows that good separation of chewing sounds from a wearer's own speech and the speech of others can be achieved by evaluating spectral features of sound signals.

### Example 2: Spatial Separation of Sound Components

An individual was fitted with an ear-worn device (MC hearing aid) including front and back microphones. Sounds were recorded using both microphones of the individual eating an apple, the individual talking, and another person talking approximately 1 meter away from the microphone. These sounds were then digitally mixed to form a test sound signal.

A correlation of the signals from the two microphones on a RIC hearing aid below 1500 Hz was then performed.

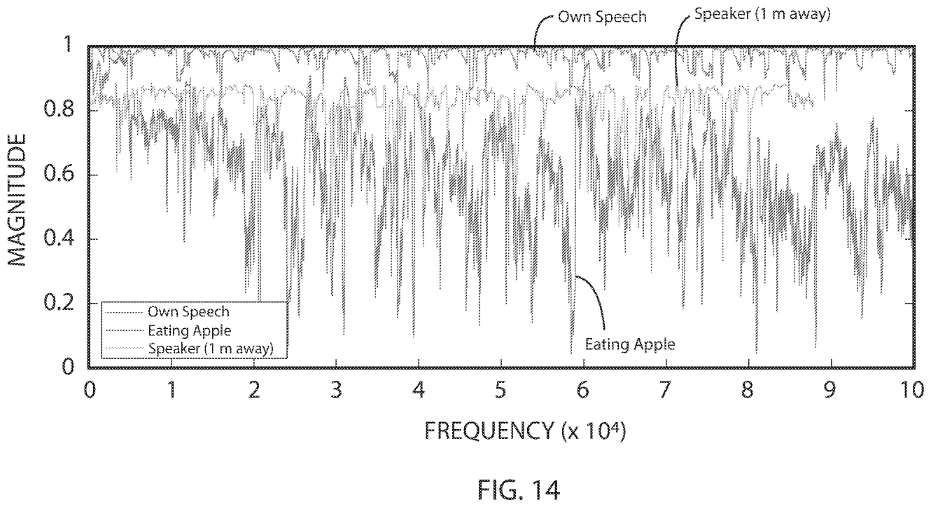

The results are shown in FIG. 14. Specifically, FIG. 14 shows separation of sound signals representing eating an apple (“Apple), versus an ear-worn device wearer's own voice (“Own Speech”), versus speech of others that was 1 meter away (“Other Speech”), using a spatial separation approach. FIG. 14 shows that good separation of chewing sounds from a wearer's own speech and the speech of others can be achieved by evaluating spatial aspects of sound signals.

### Example 3: Separation of Detected Motion

An individual was fitted with an ear-worn device including an IMU having an accelerometer. Signals from the accelerometer were then recorded while the individual was talking, then eating, then talking again after eating.

The signal was then processed by filtering out all signals below 20 Hz and then taking the square of the signal amplitude. It was found that signals above 20 Hz had a desirable signal to noise ratio for chewing.

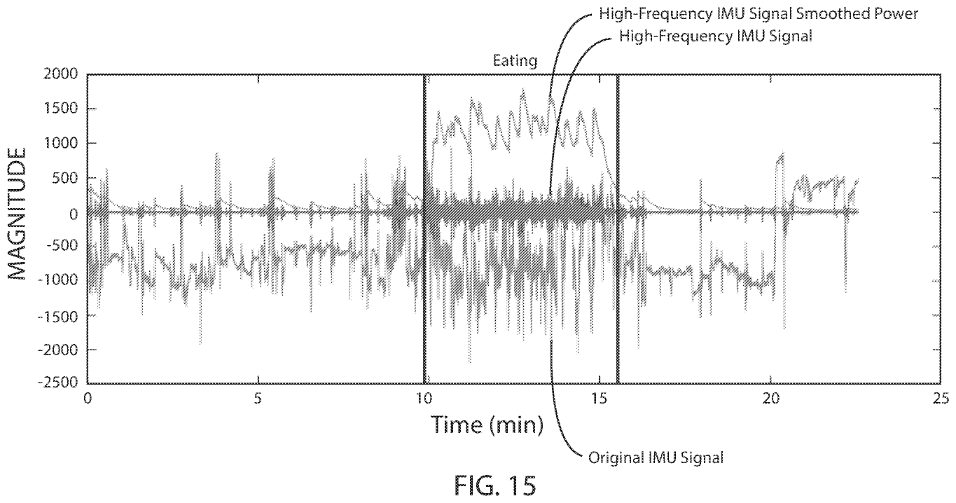

The results are shown in FIG. 15. In specific, FIG. 15 shows the signal from an IMU in its original state (“Original IMU Signal”), after filtering out low frequencies (“High-Frequency IMU Signal”), and after processing to smooth the power of the signal (“High-Frequency IMU Signal Smoothed Power”) taking the square of the signal amplitude. FIG. 15 shows that oropharyngeal events such as eating/chewing/swallowing can be readily extracted from IMU signals.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.). The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

What is claimed is:

1. An ear-worn device system comprising:
a first ear-worn device, the first ear-worn device comprising
a control circuit;
a motion sensor, wherein the motion sensor is in electrical communication with the control circuit;
at least one microphone, wherein the at least one microphone is in electrical communication with the control circuit;
an electroacoustic transducer, wherein the electroacoustic transducer is in electrical communication with the control circuit; and
a power supply circuit, wherein the power supply circuit is in electrical communication with the control circuit;
a second ear-worn device;
wherein the ear-worn device system is configured to
monitor signals from at least one of the motion sensor and the at least one microphone; and
evaluate the signals to identify oropharyngeal events.

2. The ear-worn device system of any of claims 1 and 3-43, wherein the oropharyngeal event is selected from the group consisting of mastication, swallowing, and aspiration.

3. The ear-worn device system of any of claims 1-2 and 4-43, further comprising an external device.

4. The ear-worn device system of any of claims 1-3 and 5-43, the external device comprising a smart phone.

5. The ear-worn device system of any of claims 1-4 and 6-43, wherein the external device receives data from at least one of the first ear-worn device and the second ear-worn device and evaluates the data to identify an oropharyngeal event.

6. The ear-worn device system of any of claims 1-5 and 7-43, wherein identification weighting is dependent on a current time of day.

7. The ear-worn device system of any of claims 1-6 and 8-43, wherein signal evaluation to identify oropharyngeal events includes evaluating signals from the motion sensor to identify signals between 20 and 100 Hz.

8. The ear-worn device system of any of claims 1-7 and 9-43, wherein signal evaluation to identify oropharyngeal events includes evaluating signals from the microphone to identify signals between 0 and 1.5 kHz.

9. The ear-worn device system of any of claims 1-8 and 10-43, wherein signal evaluation to identify oropharyngeal events includes evaluating signals from the microphone of the first ear-worn device and signals from a microphone of the second ear-worn device and selecting those signals emanating spatially from a location that is laterally between the first ear-worn device and the second ear-worn device.

10. The ear-worn device system of any of claims 1-9 and 11-43, wherein signal evaluation to identify oropharyngeal events includes evaluating signals from the microphone of the first ear-worn device and signals from a microphone of the second ear-worn device and selecting those signals emanating from a spatial location that is laterally between the first ear-worn device and the second ear-worn device and posterior to the lips of the ear-worn device wearer.

11. The ear-worn device system of any of claims 1-10 and 12-43, wherein the ear-worn device system is configured to evaluate the signals from the motion sensor to identify when the device wearer sits down.

12. The ear-worn device system of any of claims 1-11 and 13-43, wherein the ear-worn device system is configured to evaluate the signals from the motion sensor to identify when the device wearer tips their head backward.

13. The ear-worn device system of any of claims 1-12 and 14-43, wherein signal evaluation to identify oropharyngeal events includes evaluating signals from the motion sensor followed sequentially by evaluating signals from the microphone.

14. The ear-worn device system of any of claims 1-13 and 15-43, wherein signal evaluation to identify oropharyngeal events includes evaluating signals from the motion sensor to identify head or jaw movement followed sequentially by evaluating signals from the microphone to identify swallowing.

15. The ear-worn device system of any of claims 1-14 and 16-43, wherein signal evaluation to identify oropharyngeal events includes evaluating signals from the motion sensor and evaluating signals from the at least one microphone to identify mastication using signals from both sensors.

16. The ear-worn device system of any of claims 1-15 and 17-43, wherein signal evaluation to identify oropharyngeal events includes detecting a meal event based on detecting a cluster of mastication events.

17. The ear-worn device system of any of claims 1-16 and 18-43, wherein the ear-worn device system is configured to identify a skipped meal based on the absence of detecting a meal event and a time window for meals.

18. The ear-worn device system of any of claims 1-17 and 19-43, wherein the ear-worn device system is configured to calculate a meal variability score.

19. The ear-worn device system of any of claims 1-18 and 20-43, wherein signal evaluation to identify oropharyngeal events includes binary mastication detection per second.

20. The ear-worn device system of any of claims 1-19 and 21-43, the motion sensor comprising an accelerometer.

21. The ear-worn device system of any of claims 1-20 and 22-43, the motion sensor comprising a gyroscope.

22. The ear-worn device system of any of claims 1-21 and 23-43, wherein the at least one microphone is configured to be positioned within an ear canal of the ear-worn device wearer.

23. The ear-worn device system of any of claims 1-22 and 24-43, wherein the ear-worn device system is configured to distinguish bruxism from mastication.

24. The ear-worn device system of any of claims 1-23 and 25-43, wherein the ear-worn device system is configured to distinguish normal swallowing events from abnormal swallowing events.

25. The ear-worn device system of any of claims 1-24 and 26-43, wherein the ear-worn device system is configured to distinguish normal swallowing events from abnormal swallowing events based on a sequence of identified oropharyngeal events.

26. The ear-worn device system of any of claims 1-25 and 27-43, wherein the ear-worn device system is configured to distinguish normal swallowing events from abnormal swallowing events based on a timing of identified oropharyngeal events.

27. The ear-worn device system of any of claims 1-26 and 28-43, wherein the ear-worn device system is configured to generate an alert if abnormal oropharyngeal events are detected crossing a threshold value.

28. The ear-worn device system of any of claims 1-27 and 29-43, wherein the ear-worn device system is configured to generate an alert if abnormal oropharyngeal events are detected at a frequency that is increasing over time.

29. The ear-worn device system of any of claims 1-28 and 30-43, wherein the ear-worn device system is configured to generate an alert if mastication is detected after a predetermined time.

30. The ear-worn device system of any of claims 1-29 and 31-43, further comprising a geolocation sensor.

31. The ear-worn device system of any of claims 1-30 and 32-43, wherein the ear-worn device system is configured to generate a report showing geolocations of eating events.

32. The ear-worn device system of any of claims 1-31 and 33-43, wherein the ear-worn device system is configured to generate a report showing time patterns of eating events.

33. The ear-worn device system of any of claims 1-32 and 34-43, wherein the ear-worn device system is configured to generate a report showing frequency of fluid intake.

34. The ear-worn device system of any of claims 1-33 and 35-43, wherein the ear-worn device system is configured to generate an alert if the frequency of fluid intake falls below a fluid intake threshold value.

35. The ear-worn device system of any of claims 1-34 and 36-43, wherein the fluid intake threshold value is a predetermined static value.

36. The ear-worn device system of any of claims 1-35 and 37-43, wherein the fluid intake threshold value is a dynamically determined value.

37. The ear-worn device system of any of claims 1-36 and 38-43, the at least one microphone comprising:
a front microphone; and
a rear microphone.

38. The ear-worn device system of any of claims 1-37 and 39-43, wherein the first ear-worn device is configured to detect food types.

39. The ear-worn device system of any of claims 1-38 and 40-43, wherein the ear-worn device system is configured to estimate food intake quantities.

40. The ear-worn device system of any of claims 1-39 and 41-43, wherein the ear-worn device system is configured to estimate calorie intake.

41. The ear-worn device system of any of claims 1-40 and 42-43, the first ear-worn device comprising a temperature sensor.

42. The ear-worn device system of any of claims 1-41 and 43, the at least one microphone comprising an intracanal microphone.

43. The ear-worn device system of any of claims 1-42, the at least one microphone comprising a pair of intracanal microphones.

44. An ear-worn device comprising:
a first ear-worn device, the first ear-worn device comprising
a control circuit;
a motion sensor, wherein the motion sensor is in electrical communication with the control circuit;
at least one microphone, wherein the at least one microphone is in electrical communication with the control circuit;
an electroacoustic transducer, wherein the electroacoustic transducer is in electrical communication with the control circuit; and
a power supply circuit, wherein the power supply circuit is in electrical communication with the control circuit;
wherein the ear-worn device is configured to
monitor signals from at least one of the motion sensor and the at least one microphone; and
evaluate the signals to identify oropharyngeal events.

45. The ear-worn device of any of claims 44 and 46-72, wherein the oropharyngeal event is selected from the group consisting of mastication, swallowing, and aspiration.

46. The ear-worn device of any of claims 44-45 and 47-72, wherein signal evaluation to identify oropharyngeal events includes evaluating signals from the motion sensor to identify signals between 20 and 100 Hz.

47. The ear-worn device of any of claims 44-46 and 48-72, wherein signal evaluation to identify oropharyngeal events includes evaluating signals from the microphone to identify signals between 0 and 1.5 kHz.

48. The ear-worn device of any of claims 44-47 and 49-72, wherein the ear-worn device is configured to evaluate the signals from the motion sensor to identify when the device wearer sits down.

49. The ear-worn device of any of claims 44-48 and 50-72, wherein the ear-worn device is configured to evaluate the signals from the motion sensor to identify when the device wearer tips their head backward.

50. The ear-worn device of any of claims 44-49 and 51-72, wherein signal evaluation to identify oropharyngeal events includes evaluating signals from the motion sensor followed sequentially by evaluating signals from the microphone.

51. The ear-worn device of any of claims 44-50 and 52-72, wherein signal evaluation to identify oropharyngeal events includes evaluating signals from the motion sensor to identify head or jaw movement followed sequentially by evaluating signals from the microphone to identify swallowing.

52. The ear-worn device of any of claims 44-51 and 53-72, wherein signal evaluation to identify oropharyngeal events includes evaluating signals from the motion sensor and evaluating signals from the at least one microphone to identify mastication using signals from both sensors.

53. The ear-worn device of any of claims 44-52 and 54-72, wherein signal evaluation to identify oropharyngeal events includes detecting a meal event based on detecting a cluster of mastication events.

54. The ear-worn device of any of claims 44-53 and 55-72, wherein signal evaluation to identify oropharyngeal events includes binary mastication detection per second.

55. The ear-worn device of any of claims 44-54 and 56-72, the motion sensor comprising an accelerometer.

56. The ear-worn device of any of claims 44-55 and 57-72, the motion sensor comprising a gyroscope.

57. The ear-worn device of any of claims 44-56 and 58-72, wherein the at least one microphone is configured to be positioned within an ear canal of the ear-worn device wearer.

58. The ear-worn device of any of claims 44-57 and 59-72, wherein the ear-worn device is configured to distinguish bruxism from mastication.

59. The ear-worn device of any of claims 44-58 and 60-72, wherein the ear-worn device is configured to distinguish normal swallowing events from abnormal swallowing events.

60. The ear-worn device of any of claims 44-59 and 61-72, wherein the ear-worn device is configured to distinguish normal swallowing events from abnormal swallowing events based on a sequence of identified oropharyngeal events.

61. The ear-worn device of any of claims 44-60 and 62-72, wherein the ear-worn device is configured to distinguish normal swallowing events from abnormal swallowing events based on a timing of identified oropharyngeal events.

62. The ear-worn device of any of claims 44-61 and 63-72, wherein the ear-worn device is configured to generate an alert if abnormal oropharyngeal events are detected crossing a threshold value.

63. The ear-worn device of any of claims 44-62 and 64-72, wherein the ear-worn device is configured to generate an alert if abnormal oropharyngeal events are detected at a frequency that is increasing over time.

64. The ear-worn device of any of claims 44-63 and 65-72, further comprising a geolocation sensor.

65. The ear-worn device of any of claims 44-64 and 66-72, wherein the ear-worn device is configured to generate a report showing geolocations of eating events.

66. The ear-worn device of any of claims 44-65 and 67-72, wherein the ear-worn device is configured to generate a report showing time patterns of eating events.

67. The ear-worn device of any of claims 44-66 and 68-72, wherein the ear-worn device is configured to generate a report showing frequency of fluid intake.

68. The ear-worn device of any of claims 44-67 and 69-72, wherein the ear-worn device is configured to generate an alert if the frequency of fluid intake falls below a fluid intake threshold value.

69. The ear-worn device of any of claims 44-68 and 70-72, wherein the fluid intake threshold value is a predetermined static value.

70. The ear-worn device of any of claims 44-69 and 71-72, wherein the fluid intake threshold value is a dynamically determined value.

71. The ear-worn device of any of claims 44-70 and 72, wherein the ear-worn device is configured to generate an alert if mastication is detected after a predetermined time.

72. The ear-worn device of any of claims 44-71, the at least one microphone comprising:
a front microphone; and
a rear microphone.

73. An ear-worn device system comprising:
a first ear-worn device, the first ear-worn device comprising
a control circuit;
a motion sensor, wherein the motion sensor is in electrical communication with the control circuit;
at least one microphone, wherein the at least one microphone is in electrical communication with the control circuit;
an electroacoustic transducer, wherein the electroacoustic transducer is in electrical communication with the control circuit; and
a power supply circuit, wherein the power supply circuit is in electrical communication with the control circuit;
wherein the ear-worn device system is configured to
monitor signals from at least one of the motion sensor and the at least one microphone; and
transfer data representing the signals to an external device for identification of oropharyngeal events.

74. The ear-worn device system of any of claims 73 and 75-104, wherein the oropharyngeal event is selected from the group consisting of mastication, swallowing, and aspiration.

75. The ear-worn device system of any of claims 73-74 and 76-104, wherein signal evaluation to identify oropharyngeal events includes evaluating signals from the motion sensor to identify signals between 20 and 100 Hz.

76. The ear-worn device system of any of claims 73-75 and 77-104, wherein signal evaluation to identify oropharyngeal events includes evaluating signals from the microphone to identify signals between 0 and 1.5 kHz.

77. The ear-worn device system of any of claims 73-76 and 78-104, wherein the ear-worn device system is configured to evaluate the signals from the motion sensor to identify when the device wearer sits down.

78. The ear-worn device system of any of claims 73-77 and 79-104, wherein the ear-worn device system is configured to evaluate the signals from the motion sensor to identify when the device wearer tips their head backward.

79. The ear-worn device system of any of claims 73-78 and 80-104, wherein signal evaluation to identify oropharyngeal events includes evaluating signals from the motion sensor followed sequentially by evaluating signals from the microphone.

80. The ear-worn device system of any of claims 73-79 and 81-104, wherein signal evaluation to identify oropharyngeal events includes evaluating signals from the motion sensor to identify head or jaw movement followed sequentially by evaluating signals from the microphone to identify swallowing.

81. The ear-worn device system of any of claims 73-80 and 82-104, wherein signal evaluation to identify oropharyngeal events includes evaluating signals from the motion sensor and evaluating signals from the at least one microphone to identify mastication using signals from both sensors.

82. The ear-worn device system of any of claims 73-81 and 83-104, wherein signal evaluation to identify oropharyngeal events includes detecting a meal event based on detecting a cluster of mastication events.

83. The ear-worn device system of any of claims 73-82 and 84-104, wherein signal evaluation to identify oropharyngeal events includes binary mastication detection per second.

84. The ear-worn device system of any of claims 73-83 and 85-104, the motion sensor comprising an accelerometer.

85. The ear-worn device system of any of claims 73-84 and 86-104, the motion sensor comprising a gyroscope.

86. The ear-worn device system of any of claims 73-85 and 87-104, wherein the at least one microphone is configured to be positioned within an ear canal of the ear-worn device wearer.

87. The ear-worn device system of any of claims 73-86 and 88-104, wherein the ear-worn device system is configured to distinguish bruxism from mastication.

88. The ear-worn device system of any of claims 73-87 and 89-104, wherein the ear-worn device system is configured to distinguish normal swallowing events from abnormal swallowing events.

89. The ear-worn device system of any of claims 73-88 and 90-104, wherein the ear-worn device system is configured to distinguish normal swallowing events from abnormal swallowing events based on a sequence of identified oropharyngeal events.

90. The ear-worn device system of any of claims 73-89 and 91-104, wherein the ear-worn device system is configured to distinguish normal swallowing events from abnormal swallowing events based on a timing of identified oropharyngeal events.

91. The ear-worn device system of any of claims 73-90 and 92-104, wherein the ear-worn device system is configured to generate an alert if abnormal oropharyngeal events are detected crossing a threshold value.

92. The ear-worn device system of any of claims 73-91 and 93-104, wherein the ear-worn device system is configured to generate an alert if abnormal oropharyngeal events are detected at a frequency that is increasing over time.

93. The ear-worn device system of any of claims 73-92 and 94-104, wherein the ear-worn device system is configured to generate an alert if mastication is detected after a predetermined time.

94. The ear-worn device system of any of claims 73-93 and 95-104, further comprising a geolocation sensor.

95. The ear-worn device system of any of claims 73-94 and 96-104, wherein the ear-worn device system is configured to generate a report showing geolocations of eating events.

96. The ear-worn device system of any of claims 73-95 and 97-104, wherein the ear-worn device system is configured to generate a report showing time patterns of eating events.

97. The ear-worn device system of any of claims 73-96 and 98-104, wherein the ear-worn device system is configured to generate a report showing frequency of fluid intake.

98. The ear-worn device system of any of claims 73-97 and 99-104, wherein the ear-worn device system is configured to generate an alert if the frequency of fluid intake falls below a fluid intake threshold value.

99. The ear-worn device system of any of claims 73-98 and 100-104, wherein the fluid intake threshold value is a predetermined static value.

100. The ear-worn device system of any of claims 73-99 and 101-104, wherein the fluid intake threshold value is a dynamically determined value.

101. The ear-worn device system of any of claims 73-100 and 102-104, wherein the alert comprises a prompt for the device wearer to drink fluids.

102. The ear-worn device system of any of claims 73-101 and 103-104, wherein the alert comprises a prompt for a third-party to administer fluids to the device wearer.

103. The ear-worn device system of any of claims 73-102 and 104, the external device comprising a smart phone.

104. The ear-worn device system of any of claims 73-103, the at least one microphone comprising:
a front microphone; and
a rear microphone.

105. A method of detecting oropharyngeal events with an ear-worn device comprising:
monitoring signals from at least one of a motion sensor associated with the ear-worn device and a microphone associated with the ear-worn device; and
evaluating the signals to identify an oropharyngeal event.

106. The method of detecting oropharyngeal events with an ear-worn device of any of claims 105 and 107-116, wherein evaluating the signals to identify an oropharyngeal event further comprises evaluating signals of both the motion sensor and the microphone.

107. The method of detecting oropharyngeal events with an ear-worn device of any of claims 105-106 and 108-116, further comprising processing the signals.

108. The method of detecting oropharyngeal events with an ear-worn device of any of claims 105-107 and 109-116, wherein processing the signals further comprises filtering out motion sensor signals below 20 Hz.

109. The method of detecting oropharyngeal events with an ear-worn device of any of claims 105-108 and 110-116, wherein processing the signals further comprises correlating signals from at least two microphones to extract spatially defined signals.

110. The method of detecting oropharyngeal events with an ear-worn device of any of claims 105-109 and 111-116, wherein the spatially defined signals comprise those with a determined point of origin within the ear-worn device wearer.

111. The method of detecting oropharyngeal events with an ear-worn device of any of claims 105-110 and 112-116, wherein processing the signals further comprises exacting at least one spectral feature of the microphone signal.

112. The method of detecting oropharyngeal events with an ear-worn device of any of claims 105-111 and 113-116, wherein processing the signals further comprises exacting at least one temporal feature of the microphone signal.

113. The method of detecting oropharyngeal events with an ear-worn device of any of claims 105-112 and 114-116, wherein processing the signals further comprises filtering out signals corresponding to the voice of the ear-worn device wearer and the voices of third parties.

114. The method of detecting oropharyngeal events with an ear-worn device of any of claims 105-113 and 115-116, further comprising calculating a trend based on identified oropharyngeal events.

115. The method of detecting oropharyngeal events with an ear-worn device of any of claims 105-114 and 116, further comprising issuing an alert if an abnormal oropharyngeal event is identified.

116. The method of detecting oropharyngeal events with an ear-worn device of any of claims 105-115, wherein the oropharyngeal event comprises mastication.
